(19) United States
(12) Patent Application Publication
(10) Pub. No.: US 2015/0359921 A1
(43) Pub. Date: Dec. 17, 2015

(54) System and Method For Reducing Airborne Contamination

(71) Applicant: American Innovative Research Corp., Andover, MA (US)

(72) Inventor: David W. Palmer, Andover, MA (US)

(21) Appl. No.: 14/736,911

(22) Filed: Jun. 11, 2015

Related U.S. Application Data
(60) Provisional application No. 62/011,807, filed on Jun. 13, 2014.

(51) Int. Cl. A61L 9/20; B01D 53/00

(52) U.S. Cl. CPC A61L 9/20; B01D 53/007; B01D 2259/804

(57) ABSTRACT

A system for reducing airborne contamination includes a housing defining the structure of the system and configured to fit within a window of a building. The system also includes a variable-speed fan, and a microprocessor in communication with the fan and configured to control the speed of the fan. Within the housing, the system may include an electrical chassis that defines a chamber and supports at least some of the system's electrical components within the housing. A removable cartridge may be selectively coupled with the electrical chassis to form a germicidal radiation chamber within the housing and within an airflow path through the system. The removable cartridge includes UV light source(s) and filter(s) that sterilize the air as it passes through the system and the germicidal radiation chamber.

### PRIORITY

This application claims priority to provisional application Ser. No. 62/011,807, filed Jun. 13, 2014, entitled “System and Method for Reduced Airborne Contamination,” assigned attorney docket number 3116/107, and naming David W. Palmer as inventor, the disclosure of which is hereby incorporated by reference in its entirety.

### FIELD OF THE INVENTION

The invention generally relates to improving the air quality within a home and, more particularly, to the management and cleaning of air flow in or out of a closed space to displace contaminated air and/or produce a constant positive or negative room air pressure.

### SUMMARY OF THE INVENTION

In accordance with one embodiment of the present invention, a method for reducing airborne contamination within a home may include installing an air-pressure control system within a window of the home, and drawing air from outside the home. The air-pressure control system may include a system inlet, a system outlet, a variable speed fan configured to operate at a speed, and a motor controller in communication with the fan and configured to control the speed of the fan. The system may also include a solid state anemometer configured to monitor an air pressure differential between the system inlet and the system outlet, a closed-loop controller in communication with the motor controller and the solid state anemometer, and a germicidal radiation chamber. The closed-loop controller is configured to vary the speed of the fan based on the pressure differential between the inlet and outlet of the system. The germicidal radiation chamber may be located within an airflow path in the air-pressure control system, and may include at least one UV light source.

As the air is drawn from outside the home, it may be drawn through the system inlet and the airflow path, and the germicidal radiation chamber may sterilize the air as it passes through the airflow path. The method may then introduce (e.g., at a flowrate between 20 cubic feet per minute and 75 cubic feet per minute) the sterilized air into the home through the system outlet. The sterilized air may displace an equal volume of contaminated air within the home.

In accordance with some embodiments, the air-pressure control system may include at least one filter located within the airflow path. The filter may clean the drawn air by removing particulates from the drawn air as it passes through the filter. The filter may be located at a first end of the germicidal radiation chamber. The air-pressure control system further may also include a second filter located at a second end of the germicidal radiation chamber.

The air-pressure control system (e.g., via the germicidal radiation chamber and filter) may remove volatile organic compounds from the drawn air, and the introduced air may contain substantially no particles (e.g., dust mites, animal dander, bacteria, lead paint, household dust, cooking smoke and grease, wood and tobacco smoke, and smog) between 5.0 microns and 0.3 microns. Additionally, displacing the contaminated air within the home may improve the air quality within the home. The contaminated air may include volatile organic compounds, airborne micro-contamination, harmful gases, dust mites, animal dander, bacteria, lead paint, household dust, cooking smoke and grease, wood and tobacco smoke, and/or smog.

In some embodiments, the air-pressure control system may include an adjustable frame that, in turn, includes a top rail and a first and second adjustable side rail. Installing the air pressure control system within the window may include (1) inserting the air-pressure control system into the window such that the adjustable frame is within the window frame, (2) and adjusting the first and second side rails to expand the adjustable frame to fit the window frame. The adjustable frame may include a tape measure having a zero point located on a center point of the top rail.

The tape measure may include a first set of increasing numbers and a second set of increasing numbers. The first set of increasing numbers may increase to the left of the zero point, and the second set of increasing numbers may increase to the right of the zero point. When the first and second side rails are adjusted to expand the adjustable frame, the numbers from the first and second set of increasing numbers may be exposed. Installing the system may also include aligning the zero point with a center of the window frame, and adjusting first and second side rails such that the exposed numbers from the first and second set of increasing numbers match.

In further embodiments, the method may include providing a first and second cover for the adjustable frame, cutting the first and second covers based upon the exposed numbers from the first and second set of increasing numbers, and installing the first and second covers into the adjustable frame. The first cover may cover a first space between a side of the air-pressure control system and the first side rail. The second cover may cover a second space between an opposing side of the air-pressure control system and the second side rail.

The air-pressure control system may also include a thermostat and a thermoelectric device configured to adjust the temperature of the air drawn into the air-pressure control system. Additionally or alternatively, the air-pressure control system may include a thermocouple located within the germicidal radiation chamber and a heater located upstream of the germicidal radiation chamber. The thermocouple may be connected to the closed-loop controller and may be configured to measure a temperature of the air passing through the germicidal radiation chamber. The closed-loop controller may adjust the power to the main heater based upon the measured temperature to heat the air passing through the air-pressure control system.

In some embodiments, the air-pressure control system may also include a humidistat located between the system inlet and the system outlet, and connected to the thermoelectric device. The humidistat may be configured to measure the humidity of the drawn air. The thermoelectric device may be configured to dehumidify the drawn air based upon the measured humidity.

In accordance with further embodiments, a system for reducing airborne contamination within a home may include a housing defining the structure of the system, and an adjustable frame extending around the housing. The housing may be configured to fit within a window of the home, and the adjustable frame may be configured to expand to at least one dimension of the window. The system may also include a system inlet, a system outlet, a variable-speed fan configured to operate at a speed, and a motor controller in communication with the fan and configured to control the speed of the fan.

Some embodiments of the system may also include a solid state anemometer, a closed-loop controller, and a germicidal radiation chamber. The solid state anemometer may be configured to monitor an air pressure differential between the system inlet and the system outlet. The closed-loop controller may be in communication with the motor controller and the solid state anemometer, and may be configured to vary the speed of the fan based on the pressure differential between the inlet and outlet of the system. The germicidal radiation chamber may be located within an airflow path in the system, and may include at least one UV light source. The germicidal radiation chamber may be configured to sterilize the air as it passes through the airflow path. The sterilized air may displace an equal volume of contaminated air within the home.

The system may include at least one filter located within the airflow path (e.g., at a first end of the germicidal radiation chamber) that cleans the drawn air by removing particulates from the drawn air as it passes through the filter. Additionally or alternatively, the system may also include a second filter located at a second end of the germicidal radiation chamber. The system may be configured to introduce the sterilized air into the home at a flowrate between 20 cubic feet per minute and 75 cubic feet per minute, and/or may remove volatile organic compounds from the drawn air. The air exiting the system may contain substantially no particles (e.g., dust mites, animal dander, bacteria, lead paint, household dust, cooking smoke and grease, wood and tobacco smoke, and smog) between 5.0 microns and 0.3 microns. The sterilized air displacing the contaminated air within the home may improve the air quality within the home. The contaminated air may include volatile organic compounds, airborne micro-contamination, harmful gases, dust mites, animal dander, bacteria, lead paint, household dust, cooking smoke and grease, wood and tobacco smoke, and/or smog.

In some embodiments, the adjustable frame may include a top rail extending along a top surface of the housing, a first adjustable side rail located on a first side of the housing, and a second adjustable side rail located on a second side of the housing. The first and second adjustable rails may be configured to expand outwardly from the system such that the adjustable frame fits the window frame. The adjustable frame may include a tape measure having a zero point located on a center point of the top rail, a first set of increasing numbers, and a second set of increasing numbers. The first set of increasing numbers may increase to the left of the zero point, and the second set of increasing numbers may increase to the right of the zero point. The numbers from the first and second set of increasing numbers may be exposed as the first and second adjustable rails are expanded outwardly. When the system is installed in the window, the zero point may be aligned with a center of the window frame.

The adjustable frame may also include a first cover configured to cover a space between the first side of the housing and the first adjustable side rail, and a second cover configured to cover a space between the second side of the housing and the second adjustable side rail. The first and second covers may be sized based on the exposed numbers on the tape measure.

In further embodiments, the system may include a thermostat and a thermoelectric device configured to adjust the temperature of the air drawn into the system based upon a signal from the thermostat. Additionally or alternatively, the system may include a heater located upstream of the germicidal radiation chamber, and a thermocouple located within the germicidal radiation chamber. The thermocouple may be connected to the closed-loop controller and may be configured to measure the temperature of the air passing through the germicidal radiation chamber. The closed-loop controller may adjust the power to the main heater based upon the measured temperature to heat the air passing through the air-pressure control system. The system may also include a humidistat that may be located between the system inlet and the system outlet, and may be configured to measure the humidity of the drawn air. The thermoelectric device may be connected to the humidistat and may be configured to dehumidify the drawn air based upon the measured humidity.

In still further embodiments, the airflow path may be blackened to prevent UV reflection through the system inlet and system outlet. The closed-loop controller may include a microprocessor configured to compare an output from the solid state anemometer and a setpoint value, and adjust the speed of the fan based on the difference between the solid state anemometer output and the setpoint value. The system may also include a safety sensor in communication with the microprocessor. The microprocessor may alarm when the system is not operating at the setpoint values. Upon a change in condition within the home, the closed-loop controller may bring the fan to full speed, and then reduce the speed of the fan to obtain a setpoint value.

In accordance with additional embodiments, a system for reducing airborne contamination within a building includes a housing defining the structure of the system, a system inlet and a system outlet. The system may also include a variable-speed fan configured to operate at a speed, and a microprocessor in communication with the fan and configured to control the speed of the fan. An electrical chassis located within the housing may define a chamber, and support at least some of the system's electrical components within the housing. The housing may be configured to fit within a window of the building.

The system may also include a removable cartridge that may be selectively coupled with the electrical chassis to form a germicidal radiation chamber within the housing and located within an airflow path through the system. The removable cartridge may include at least one UV light source and at least one filter. The UV light source(s) and the filter(s) may sterilize air as it passes through the system and the germicidal radiation chamber. The removable cartridge may also include a first electrical connector that connects with a second electrical connector located on the electrical chassis (e.g., when the cartridge is coupled with the chassis). The first and second electrical connectors may electrically connect the microprocessor and the at least one UV lamp. The microprocessor may control the power level of the UV lamp(s).

In some embodiments, the housing may include a removable bezel that is connected to the housing via a magnet. The system may also include a hall-effect transistor, and the magnetic field created by the magnet may energize the hall-effect transistor when the bezel is connected to the housing. Additionally, removal of the removable bezel may turn off the system (e.g., by de-energizing the hall effect transistor). The germicidal radiation chamber may include a perforated baffle at an input end of the chamber. Similarly the removable cartridge may include a perforated baffle. The perforated baffle(s) may include a titanium oxide coating.

The system may also include a temperature transducer and a humidistat located at the system outlet. The temperature transducer may be configured to measure the temperature of the air exiting the system, and the humidistat may be configured to measure the humidity of the air exiting the system. The microprocessor may be in electrical communication with the temperature transducer and the humidistat, and may control the system operation to maintain comfortable living conditions within the building (e.g., based on the measured temperature and/or the measured humidity). For example, the system can include a resistive heater, and one or more Peltier modules (e.g., a first Peltier module configured to cool incoming air and a second Peltier module configured to dehumidify incoming air). The microprocessor may be configured to control the duty cycle power to the resistive heater and/or the power to first and/or second Peltier module.

The microprocessor may also be configured to monitor the power consumption of the system and compare the monitored power consumption with a stored value to validate system functionality. The control panel may have a display, and the microprocessor may send a message to the control panel indicating that the system is operating out of specifications (e.g., if a signal level of an air velocity sensor is substantially different from stored value and/or the power consumption of the system is substantially not equal to the stored value). Additionally or alternatively, the microprocessor may (1) monitor the total runtime of the system and send a change cartridge message to the control panel if the total runtime exceeds a threshold value, and/or (2) monitor a power of level of the fan and determine, based at least in part on the power level of the fan, if the at least one filter is clogged.

In some embodiments, the system may include a mounting kit that allows the housing to be secured to a wall of the building. For example, the mounting kit may include a body portion that supports the housing, and a divided tube that may extend through an opening within the wall of the building. The divided tube may include an air inlet pathway and an exhaust pathway. The air inlet pathway may be configured to allow the variable speed fan to draw air from an outside atmosphere through the air inlet pathway and into the system inlet. The exhaust pathway may be configured to allow the system to send exhaust air to the outside atmosphere. The mounting kit may also include a dividing plate (e.g., an insulated plate) that separates the air inlet pathway from the exhaust pathway to prevent drawn air from mixing with exhaust air.

The mounting kit may include an attachment bracket that is secured to a surface of the housing, and may be used to attach the housing to the body portion of the mounting kit. In such embodiments, the body portion of the mounting kit may have an attachment slot, and a portion of the attachment bracket may be inserted into the attachment slot when the housing is secured to the body portion of the mounting kit.

The body portion of the mounting kit may include a vertically extending portion and a horizontally extending portion. The vertically extending portion may extend along a portion of the wall, and the horizontally extending portion may support the housing (e.g., the housing may rest on the horizontally extending portion). In some embodiments, the vertically extending portion may include mounting holes that allow the body portion of the mounting kit to be secured to the wall. The horizontally extending portion may form a condensate tray, and the mounting kit may include an edge gasket that extends along at least a portion of the body portion and seals against the housing.

In accordance with further embodiments, a method for reducing airborne contamination within a building may include installing an air purification system within an opening (e.g., a window or opening through a wall) in the building, drawing air from outside the building through the system inlet and the airflow path, operating the air purification system at peak air flow, and introducing the sterilized air into the building through the system outlet. The sterilized air may displace contaminated air within the building, and the air pressure within the building may increase until the contaminated air displaced from the building equals a flow rate of the sterilized air entering the building.

The air purification system may include a housing defining the structure of the system, the system inlet, the system outlet, a variable-speed fan configured to operate at a speed, and a microprocessor in communication with the fan and configured to control the speed of the fan. Within the housing, the system may include an electrical chassis that defines a chamber and supports at least some of the system's electrical components within the housing. A removable cartridge may be selectively coupled with the electrical chassis to form a germicidal radiation chamber within the housing. The germicidal radiation chamber maybe located within the airflow path through the system and may sterilize the air as it passes through the airflow path. For example, the removable cartridge may include one or more UV light sources and at least one filter. The UV light source(s) and the filter(s) may sterilize the air as it passes through the system and the germicidal radiation chamber.

The removable cartridge may also include a first electrical connector that connects with a second electrical connector located on the electrical chassis to electrically connect the microprocessor and the at least one UV lamp. The microprocessor may then control a power level of the at least one UV lamp.

The housing may include a removable bezel, and the system may include a hall-effect transistor. The bezel may be removably connected to the housing via a magnet, and the magnetic field created by the magnet may energize the hall-effect transistor when the bezel is connected to the housing. Conversely, removal of the bezel may turn off the system.

The system may also include a temperature transducer located at the system outlet and configured to measure the temperature of the air exiting the system, and a humidistat located at the system outlet and configured to measure the humidity of the air exiting the system. In such embodiments, the method may include controlling system operations (using the microprocessor) to maintain comfortable living conditions within the building (e.g., based at least in part on the measured temperature and the measured humidity). Additionally or alternatively, the system can include a resistive heater located at the system inlet, and the method may include tempering the incoming air using the resistive heater. For example, the microprocessor may control a duty cycle power to the resistive heater.

In some embodiments, the method may also include monitoring the power consumption of the air purification system, comparing the monitored power consumption with a stored power consumption range, and validating system functionality if the monitored power consumption is within the stored power consumption range. If the power consumption of the system is not within the stored power consumption range, the method may send, using the microprocessor, a message to the control panel, and display the message on the control panel. The message may indicate that the system is operating out of specification. Similarly, the method may monitor the total runtime of the system, and send a change cartridge message to the control panel if the total runtime exceeds a threshold vale.

In additional embodiments, the air purification system may include a mounting kit that allows the housing to be secured to a wall of the building. The mounting kit may have a body portion configured to support the housing, and a divided tube that extends through the opening within the wall of the building. When installing the system, the method may include passing the divided tube through the opening in the wall of the building. The divided tube may include an air inlet pathway and an exhaust pathway. Drawing air from outside the building may include drawing air from an outside atmosphere through the air inlet pathway and into the system inlet. The exhaust pathway may be configured to allow the system to send exhaust air to the outside atmosphere. The mounting kit may also include a dividing plate that separates the air inlet pathway from the exhaust pathway to prevent drawn air from mixing with exhaust air.

The mounting kit may also include an attachment bracket that may be secured to a surface of the housing and used to attach the housing to the body portion of the mounting kit. For example, the mounting kit may have an attachment slot, and the method may include inserting at least a portion of the attachment bracket into the attachment slot to secure the housing to the body portion of the mounting kit.

The body portion of the mounting kit may include a vertically extending portion and a horizontally extending portion. The vertically extending portion may extend along a portion of the wall and may include mounting holes to allow the body portion to be secured to the wall. The horizontally extending portion may support the housing, and may form a condensate tray. The mounting kit may also have an edge gasket extending along at least a portion of the body portion to seal against the housing.

In accordance with further embodiments, a system for reducing airborne contamination within a building includes an outside housing configured to extend through a wall of the building, an inside housing configured to be located within the interior of the building, and a flexible duct extending between the outside housing and inside housing. The flexible duct may be configured to allow air to flow from the outside housing to the inside housing, and the system may include an inlet within the outside housing and an outlet in the inside housing. The system may also include a variable-speed fan that is located within the inside housing and is configured to operate at a speed to draw air into the system inlet. A microprocessor in communication with the fan may control the speed of the fan.

In some embodiments, the system may also include an electrical chassis located within the inside housing, and a removable cartridge configured to be selectively coupled with the electrical chassis to form a germicidal radiation chamber. The electrical chassis may define a chamber and may support at least some of the system's electrical components within the inside housing. The germicidal radiation chamber may be located within the inside housing and within a main airflow path through the system. The removable cartridge may include at least one UV light source and at least one filter. The UV light source(s) and the filter(s) may be configured to sterilize air as it passes through the system and the germicidal radiation chamber.

The flexible duct may include an electrical cable that is configured to electrically connect the inside housing with the outside housing. The outside housing may include a plenum dividing wall that forms a first air flow path and a secondary air flow path through the outside housing. The first air flow path may be part of the main air flow path through the system. The system inlet may be located within the first air flow path, and the variable-speed fan may be configured to draw air through the first air flow path. Additionally, the outside housing may include a cooling fan that is located within the secondary air flow path, and configured to draw air through the secondary air flow path (e.g., over the hot side of at least one Peltier module located within the outside housing).

The removable cartridge may include a first electrical connector configured to connect with a second electrical connector located on the electrical chassis when the cartridge is coupled with the chassis. The first and second electrical connectors may electrically connect the microprocessor and the at least one UV lamp. The microprocessor may control a power level of the at least one UV lamp.

The inside housing may include a bezel that is removably connected to the inside housing via a magnet. Additionally, the system may include a hall-effect transistor, and a magnetic field created by the magnet may energize the hall-effect transistor when the bezel is connected to the inside housing. In such embodiments, removal of the bezel may turn off the system. The germicidal radiation chamber may include a perforated baffle at an input end of the germicidal radiation chamber, and the removable cartridge may include a perforated baffle. The perforated baffle(s) may include a titanium oxide coating.

In further embodiments, the system may include a temperature transducer and a humidistat located at the system outlet. The temperature transducer may be configured to measure the temperature of air exiting the system. The humidistat may be configured to measure the humidity of air exiting the system. The microprocessor may be in electrical communication with the temperature transducer and the humidstat, and may be configured to control system operation to maintain comfortable living conditions within the building, based at least in part on the measured temperature and the measured humidity. The system may also include a resistive heater that is located within the outside housing, and configured to temper incoming air. The microprocessor may be configured to control the duty cycle power to the resistive heater.

Additionally, the system may include a first and second Peltier module within the outside housing. The first Peltier module may be configured to cool incoming air, and the second Peltier module may be configured to dehumidify incoming air. The microprocessor may be configured to control power to the first and/or second Peltier module. The microprocessor may also monitor the power consumption of the system and compare the monitored power consumption with a stored value to validate system functionality.

The system may also include a control panel that has a display and is located on the inside housing. The microprocessor may send a message to the control panel indicating that that the system is operating out of specifications if the power consumption of the system is substantially not equal to the stored value. The microprocessor may also monitor a total runtime of the system and send a change cartridge message to the control panel if the total runtime exceeds a threshold vale. Additionally, power of level of the fan and determine, based at least in part on the power level of the fan, if the at least one filter is clogged. Additionally the microprocessor may be configured to monitor the signal level of an air velocity sensor to determine if the signal is substantially different from stored value.

In some embodiments, the system may include a baffle located within the germicidal radiation chamber (e.g., at the input end of the radiation chamber), and a flow straightener located downstream of the baffle (e.g., also at the input end of the radiation chamber). The baffle may prevent UV radiation from exiting the germicidal radiation chamber. The baffle and/or the flow straightener may include a titanium oxide coating. The baffle may be a flat plate sized such that gaps are formed between at least one end of the flat plate and an inner wall of the germicidal radiation chamber (e.g., to allow air to pass over the flat plate).

The system may also include an air velocity sensor located between the baffle and the flow straightener. The air velocity sensor may measure the velocity of the air passing over the baffle, and may be electrically connected to the microprocessor. The microprocessor may monitor the measured air velocity and determine if a filter is clogged based upon the measured velocity.

### BRIEF DESCRIPTION OF THE DRAWINGS

FIG. 1 shows an air-pressure-control system in accordance with an embodiment of the present invention.

FIG. 2 shows an airflow diagram of the system shown in FIG. 1.

FIG. 3 shows a logic diagram of the system shown in FIG. 1.

FIG. 4 shows an exemplary germicidal radiation chamber and electrical chassis in accordance with embodiments of the present invention.

FIG. 5 shows the exemplary germicidal radiation chamber of FIG. 4 in accordance with embodiments of the present invention.

FIG. 6 shows the germicidal radiation chamber of FIG. 4 with a chamber cover and UVC sensor in accordance with embodiments of the present invention.

FIG. 7 shows the inside of the germicidal radiation chamber of FIG. 4 in accordance with embodiments of the present invention.

FIG. 8 shows the inside of the electrical chassis of FIG. 4 in accordance with embodiments of the present invention.

FIG. 9 shows another view of the internals of the exemplary electrical chassis shown in FIG. 4 in accordance with embodiments of the present invention.

FIG. 10 shows a fan assembly with pre-filter in accordance with an embodiment of the air-pressure control system.

FIG. 11 shows an exemplary control panel in accordance with embodiments of the present invention.

FIG. 12 shows an exemplary outside shell with insulation on exposed elements in accordance with embodiments of the present invention.

FIG. 13 shows an alternative embodiment of an air-pressure-control system in accordance with an embodiment of the present invention.

FIG. 14 shows an airflow diagram of the system shown in FIG. 13.

FIG. 15 shows a logic diagram of the system shown in FIG. 13.

FIG. 16 shows an exemplary electrical schematic of the air-pressure-control system shown in FIG. 13, in accordance with embodiments of the present invention.

FIG. 17 shows an exemplary germicidal radiation chamber and electrical chassis of the system shown in FIG. 13, in accordance with embodiments of the present invention.

FIG. 18 shows the air-pressure-control system shown in FIG. 13 with an adjustable frame and mounting components, in accordance with embodiments of the present invention.

FIG. 19 shows the air-pressure-control system shown in FIG. 13 with the front panel removed to show the internal filters, in accordance with embodiments of the present invention.

FIG. 20 shows the air-pressure-control system shown in FIG. 13 with the back cover removed, in accordance with embodiments of the present invention.

FIG. 21 shows another view of the internals of the air-pressure-control system shown in FIG. 13 in accordance with embodiments of the present invention.

FIG. 22 shows an additional view of the internals of the air-pressure-control system shown in FIG. 13 in accordance with embodiments of the present invention.

FIG. 23 shows a further view of the internals of the air-pressure-control system shown in FIG. 13 in accordance with embodiments of the present invention.

FIG. 24 shows a flowchart showing the steps of one method for improving the air quality within a home, in accordance with some embodiments of the present invention.

FIG. 25 shows a further view of the internals of the air-pressure-control system shown in FIG. 13 including an air-flow path through the system, in accordance with embodiments of the present invention.

FIG. 26 shows a home and the progression of clean air through the home and the displacement of contaminated air, in accordance with some embodiments of the present invention.

FIG. 27 is a chart showing the type and size typical airborne contamination within a household.

FIG. 28 shows an embodiment of an air purification system in accordance with various embodiments of the present invention.

FIG. 29 shows the air purification system of FIG. 28 with the front cover removed and reusable cartridge partially removed, in accordance with various embodiments of the present invention.

FIG. 30 shows the air purification system of FIG. 28 with the reusable cartridge removed, in accordance with various embodiments of the present invention.

FIGS. 31A and 31B schematically show an exemplary wall mounting kit in accordance with various embodiments of the present invention.

FIG. 32 schematically shows an exemplary embodiment of a two piece air purification system, in accordance with embodiments of the present invention.

FIG. 33 schematically shows an alternative embodiment of a two piece air purification system in accordance with various embodiments of the present invention.

FIG. 34 schematically shows an exemplary baffle and flow straightener configuration, in accordance with various embodiments of the present invention.

FIG. 35 schematically shows an alternative baffle and flow straightener configuration, in accordance with various embodiments of the present invention.

### DETAILED DESCRIPTION OF SPECIFIC EMBODIMENTS

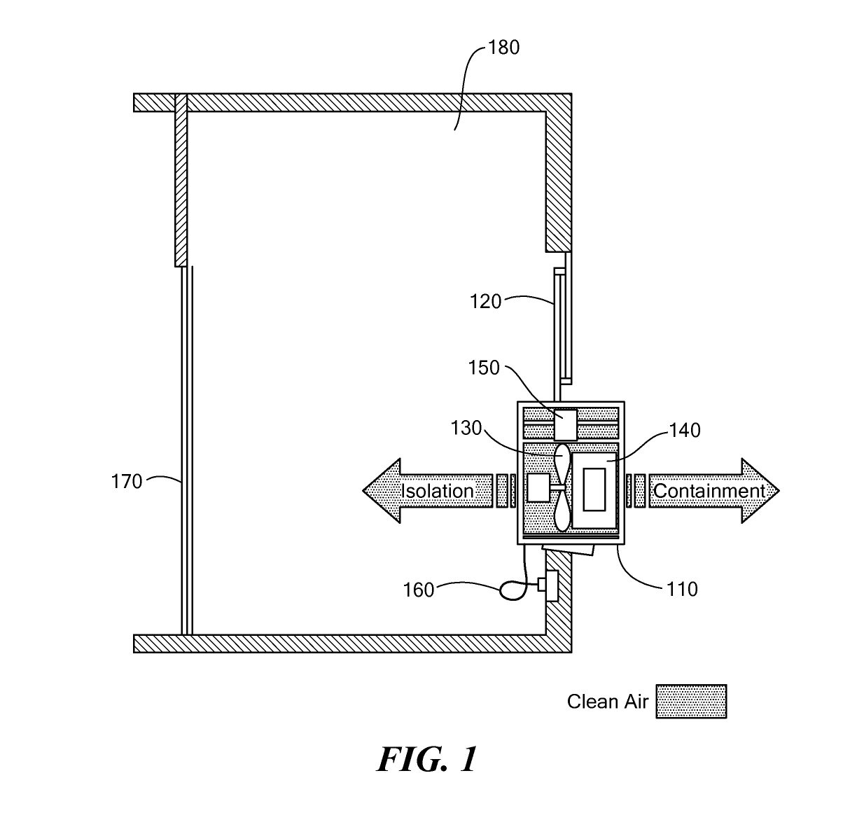

FIG. 1 shows an air-pressure-isolation system 110 in accordance with the present invention. The system 110 may be a through window, “plug and play” type system. As such, the system 110 can transform a closed space 180 into either an isolation or a containment room by placing the system 110 into a window 120 and plugging a power cord 160 into a standard wall socket. The inward facing side of the system 110 may have a stylish design so that it does not negatively impact the aesthetics of the closed space 180. The outward facing side of the system 110 may have a design that is suitable for exposure to the environment.

In an isolation configuration, a variable speed fan 130 forces clean air into the closed space 180, resulting in a positive pressure within the closed space 180. In order to produce a constant positive pressure consistent with surgical sites and clean rooms, the system 110 may control the air flow into the room, by varying the speed of the fan, to match the air flow out of the room through gaps around windows and doors. In the containment configuration, a variable-speed fan 130 forces air out of closed space 180, resulting in a negative room air pressure. In either orientation, a germicidal radiation chamber 140, located within a closed airflow path, cleans the air as it passes through system 110. If the system 110 is not installed in a window, the user can add an extension to the air path out of the germicidal radiation chamber 140 to reach the outside environment.

In some embodiments, the system 110 may contain multiple variable-speed fans. If more than one variable-speed fan is present, the fans may operate such that they force air in multiple directions.

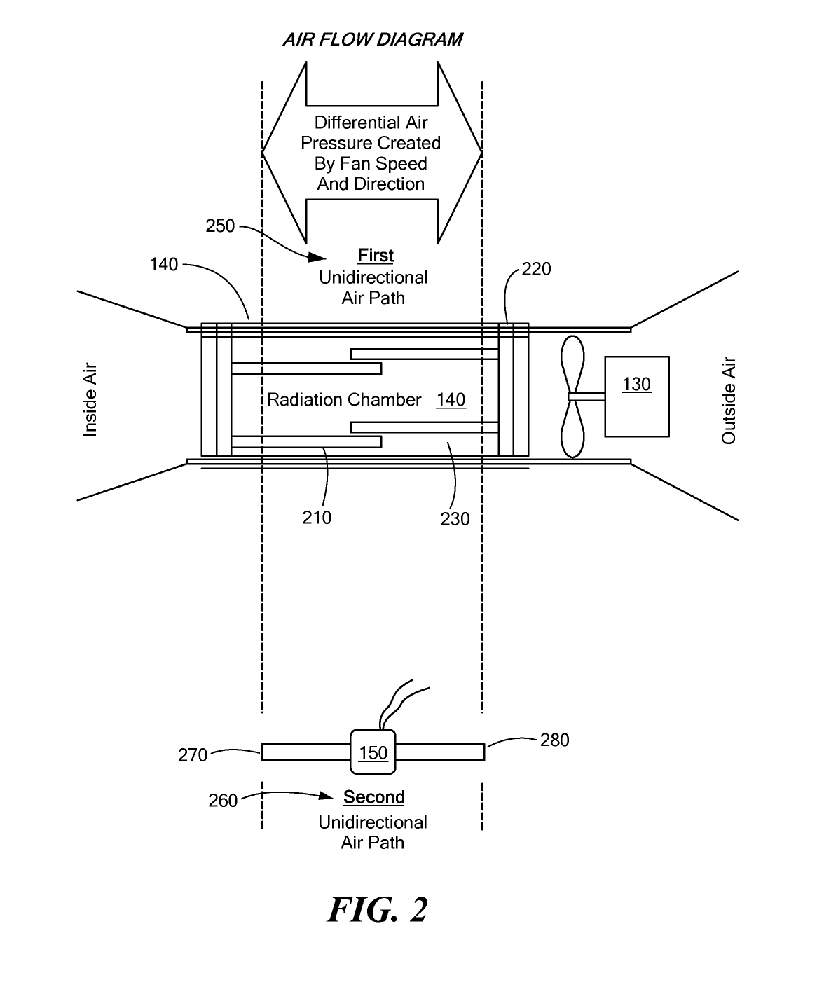

As show in FIG. 2, the germicidal radiation chamber 140 may contain ultraviolet lamps 210 that radiate at a wavelength of approximately 253.7 nanometers. UV radiation at 253.7 nanometers has been proven to inflict the greatest amount of damage on living and dormant micro-organisms. For example, at 253.7 nanometer wavelength, UV testing on influenza indicates a 90% kill ratio with severe damage (sufficient to neutralize) inflicted on the remaining 10%. The targets of the germicidal radiation chamber 140 include, but are not limited to: viruses, bacteria, fungus, mold, and spores. Although a 253.7 nanometer wavelength is used as an example, the UV wavelength can be adjusted to maximize the damage to any one species of micro-organisms.

The radiation chamber 140 may also provide access to the UV lamps 210 so that a user may replace the UV lamps 210 when needed. The user can install the UV lamps 210 from outside of the germicidal radiation chamber 140 so that they need not disassemble the chamber 140. The access to the UV lamps 210 may include a kill switch that shuts off the system 110 to prevent a user from accessing the UV lamps 210 during operation. Alternatively, the germicidal radiation chamber 140 may be a cartridge design that a user can completely remove and replace at a remote location. In some embodiments, the germicidal radiation chamber 140 may include multiple UV lamps with varying wavelengths to target different types of airborne particulates or micro-organisms.

As mentioned above, the germicidal radiation chamber 140 can be removable. In embodiments containing a removable radiation chamber 140, the system may also include an interlock switch that is electrically connected to the radiation chamber 140. The interlock switch can verify that the radiation chamber 140 is installed correctly and, in the event of incorrect installation, cut off the main power to the system 110, for example, to prevent accidental exposure to UV light.

Destruction and neutralization of micro-organisms using UV light depends on the amount of UV light that the micro-organisms are exposed to and the exposure time. To increase the amount of exposure, the inside surface of the germicidal radiation chamber 140 may contain a reflective coating 230. The reflective coating 230 reflects the UV light within the chamber, exposing the micro-organisms to greater amounts of UV light and, thus, increasing the micro-organism kill and neutralization ratios. Additionally, in some embodiments, the exposure time may be increased by slowing down the air flow within the germicidal radiation chamber 140. A laminar air flow through chamber 140 can assure that the resident time and exposure is uniform and equal throughout chamber 140. To further increase the exposure and residence time, the chamber 140 should be as large as possible within the constraints of overall size of the system 110. Dead spots in the airflow should be minimized.

UV light is hazardous and should be contained within the germicidal radiation chamber 140 and system 110. To prevent UV light from escaping and to help prevent accidental exposure to UV light, the germicidal radiation chamber 140 may include baffles 220 at one or both ends. Additionally or alternatively, the airflow path of the system 110 may be blackened to prevent UV reflection through the system inlet or outlet.

A differential-air-pressure transducer 150 can measure the air pressure at the inlet and outlet of the system 110. The differential-air-pressure transducer 150 may sample and measure the air pressure of the inside air through a closed space air port 270 and can measure the outside air pressure through an outside air port 280. The system 110 may contain pressure-tight connections between the differential pressure transducer 150 and air ports 270, 280. The outside air port 280 may contain provisions to prevent blockage from freezing weather and other variables such as insects. If the system 110 is not installed in a window, the outside air port 280 may also include an extension to reach the outside environment. In some embodiments, the differential-air-pressure sensor 150 can be a hot-wire or solid state anemometer. In other embodiments, a pressure transducer 150 may be located in a second airflow path 260. As shown in FIG. 2, the second airflow path 260 may be separate and distinct from the first airflow path 250, which contains the germicidal radiation chamber 140.

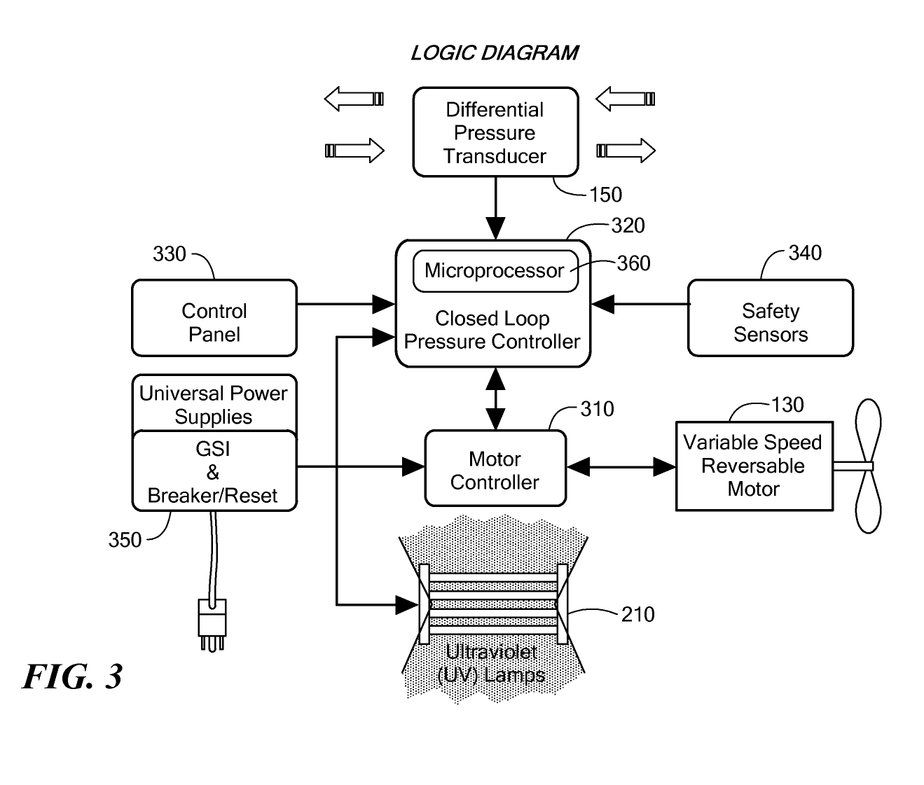

As shown in FIG. 3, the system 110 may include a closed-loop controller 320. that is connected to the differential-air-pressure transducer 150, and a motor controller 310. The closed-loop controller 320 may monitor the pressure differential between the system inlet and the system outlet and, based on the pressure differential, adjust the speed of the fan 130 via the motor controller 310. By controlling the speed of the fan 130 via the motor controller 310, the closed-loop controller 320 is able to control the pressure within the closed space 180. The motor controller 310 may work on all voltages and cycles, and have a selectable voltage switch. In embodiments containing multiple fans, the motor controller 310 may have a different controller power situation for each unit.

During startup, the closed-loop controller 320 may be configured to expect a worst case scenario and bring the fan 130 to full speed. In response to a power interruption to the system 110, the closed-loop controller 320 may provide an orderly shut down and start up process.

The closed-loop controller 320 may include a microprocessor 360. The microprocessor 360 may compare the differential-air-pressure transducer 150 output to a setpoint inputted by the user via a control panel 330 (discussed below) or pre-programmed into the system 110. The microprocessor 360 may then adjust the speed of the fan 130 to maintain the pressure within the closed space 180 at the setpoint value. When the system 110 is operating out of set point conditions, the closed-loop controller 320 may trigger an alarm to alert the user/home owner.

The closed-loop controller 320 may also include a second control band capable of recognizing when a door 170 (FIG. 1) is opened or when another change in condition within the space 180 occurs. The closed-loop controller 320 may then respond to such a condition by taking the fan 130 to full speed and then closing on a setpoint. The closed-loop controller 320 may also set a dead band to prevent the fan 130 from hunting.

In other embodiments, the closed-loop controller 320 may verify the presence of UV light and control the intensity of the UV radiation based on the air flow through the system 110. For example, the closed-loop controller 320 may control the intensity of the UV radiation by turning on all UV lamps 210 for maximum radiation, or by turning on one UV lamp at a time to perform a step function of radiation levels. The closed-loop controller 320 may also recognize if a UV lamp fails and switch the power to a functioning lamp.

In some embodiments of the present invention, the closed-loop controller 320 may contain a software port (not shown). The software port allows a user to download new software revisions (including new/updated setpoint values) and to test individual functions of the system 110.

In further embodiments, the system 110 may contain a control panel 330 that, among other things, allows a user to input setpoints values into the control panel 330. The control panel 330 may also contain a switch (not shown) to allow the user to choose between either positive or negative room pressure. The switch can be either a mechanical switch, a key pad, or a key pad multiple digital code. In embodiments containing multiple fans, the control panel 330 may allow the user to select one of the fans to move in a different direction. Other functions of the control panel 330 include, but are not limited to, diagnosing one or all functions of the control system, and displaying when routine services, such as UV lamp 210 replacements, are needed. The control panel 330 may be available in multiple languages.

In accordance with other embodiments of the present invention, the system 110 may also contain safety sensors 340. The safety sensors 340 may include an audible or visible alarm. The safety sensor 340 and the associated alarm may be in communication with the microprocessor 360 and the closed-loop controller 320. After receiving a signal from the closed-loop controller 320, the safety sensor 340 may trigger the alarm if the system 110 is not operating at the setpoint value or when system components are not functioning properly.

A universal power supply 350 supplies power to the system 110. The power supply 350 contains a GSI and a breaker reset and may be plugged into a standard wall socket.

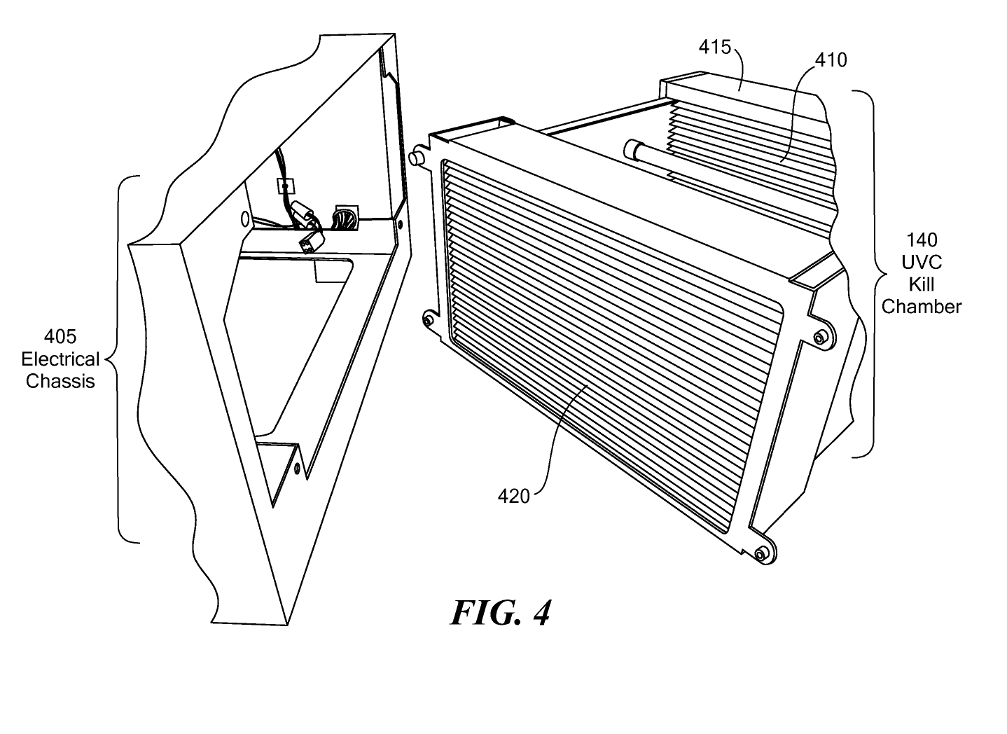

As shown in FIG. 4, the germicidal radiation chamber 140 can be contained within an electrical chassis 405. In such embodiments, a user can essentially slide the germicidal radiation chamber 140 into the electric chassis 405 to create the complete system 110. As discussed in greater detail below, the electrical chassis 405 houses many of the electrical and mechanical components of the system 110.

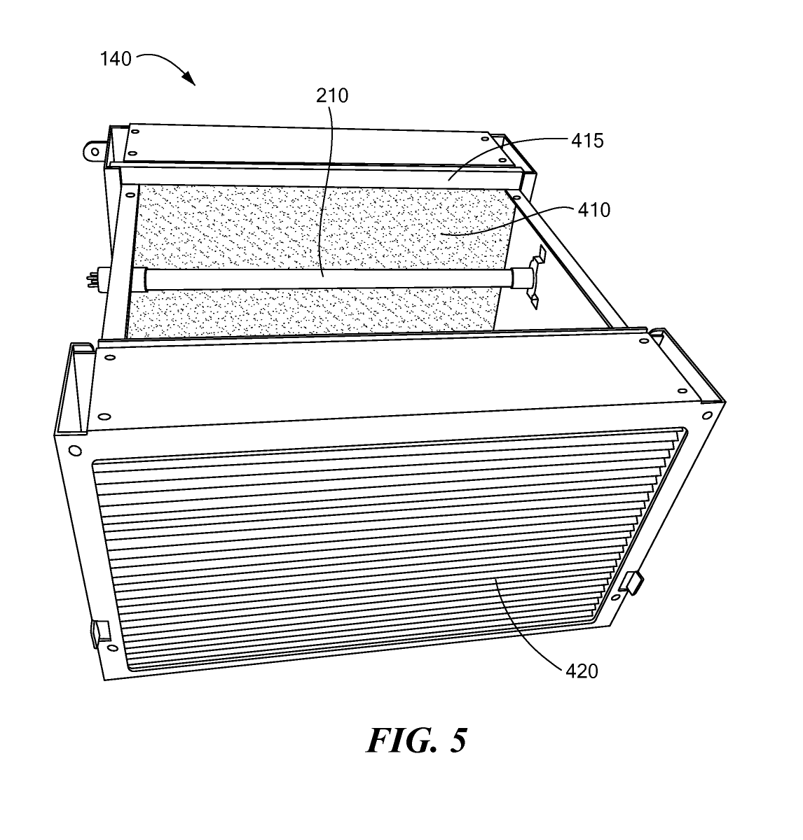

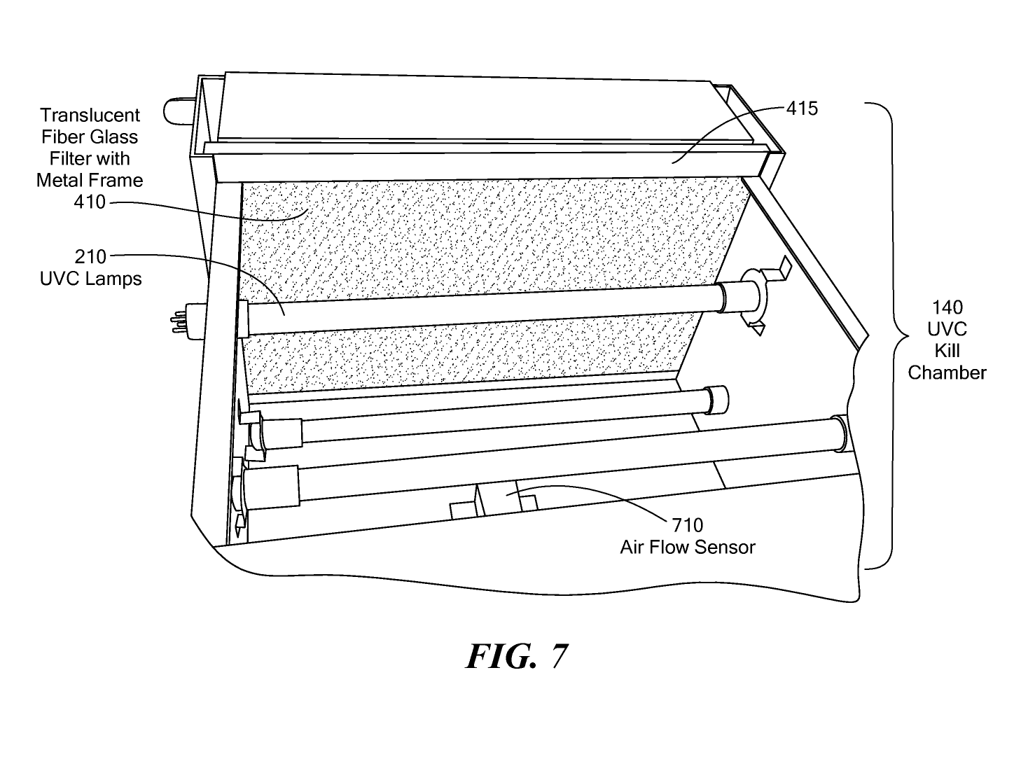

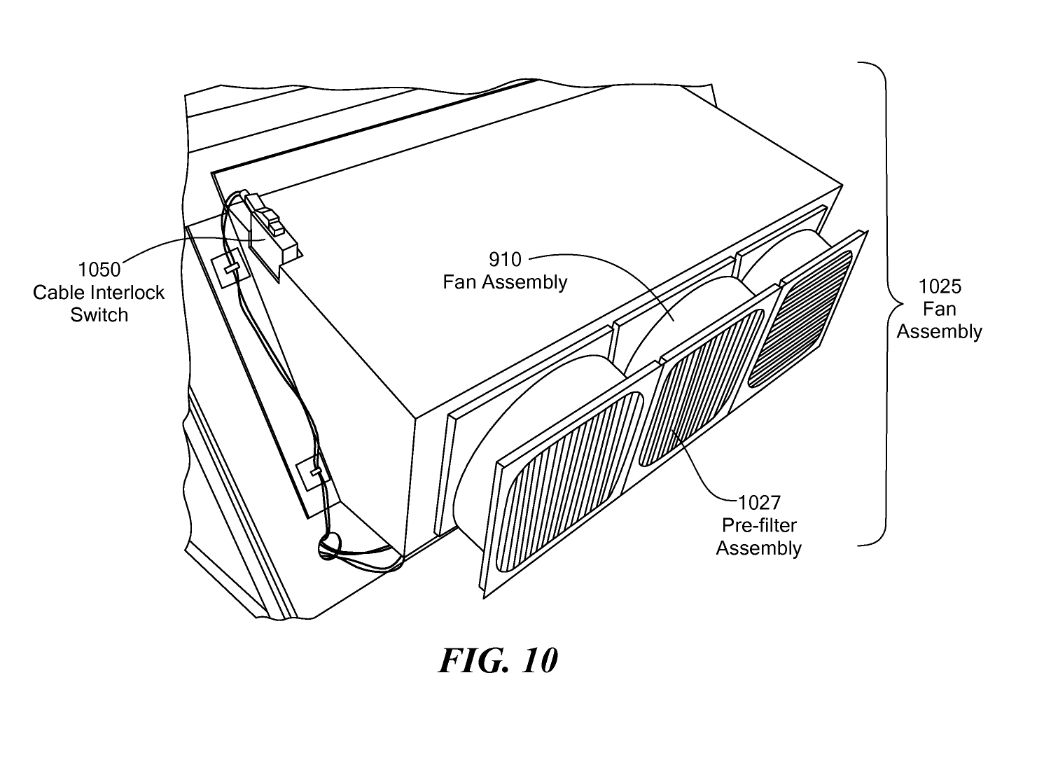

The system 110 may be a filter-less system or may include a HEPA filter 410. In filter-less embodiments, the UV light kills or neutralizes the micro-organisms as they pass through the germicidal radiation chamber 140. As shown in FIGS. 4 and 5, in systems 110 having filters, the HEPA filter 410 may be located at one or both ends of the germicidal radiation chamber 140. For example, the filter 410 may be at the opposite end of the germicidal radiation chamber 140 from the fans 910 (see FIGS. 7 and 10). To ease filter installation and replacement, the germicidal radiation chamber 140 may include slots that allow access to the filter 410. The addition of the filter 410 and two more sensors (an air flow sensor in the UVC chamber and a UVC level sensor in the UVC chamber, discussed in greater detail below) essentially make the system 110 a portable air cleaner and air sterilizer as well as a room isolation controller and a room containment controller.

In preferred embodiments, the filter 410 should be a translucent fiber glass HEPA filter. The translucent filter allows the UV radiation to pass through the filter, allowing the UVC radiation to kill the viruses as they move through the germicidal radiation chamber 140 and pass through the filter 410. In some embodiments, the filter may be pleated to increase the effective surface area of the filter. The pleated filters can be oriented such that the pleats are vertical, and the axis of the UV lamp 210 is transverse to the filter pleat axis. In preferred embodiments, the UV lamps 210 are co-planar.

The HEPA filter 410 will trap larger contamination, exposing the larger contamination to continuous irradiation by the high intensity UVC lamps 210. By doing so, the filter 410 allows for destruction of the larger particulates (which require greater amounts of irradiation to be killed), while maintaining a manageable system size and the flowrates needed for room isolation and containment. It is important to note that the UVC radiation will dissociate most organic particulates from the HEPA filter 410, creating a self-cleaning filter.

The filter 410 and filter frame 415 (FIG. 7) should be constructed from materials that are resistant to UVC radiation. For example, the filter 410 may be translucent fiber glass, and the filter frame 415 may be metal.

The entrance to the germicidal radiation chamber 140 can also include a UVC light baffle and a flow straightener 420. As discussed above, the UVC light baffles prevent UV light from exiting the germicidal radiation chamber 140. As the name suggests, the flow straightener(s) 420 straighten the air flow through the system and may be used to reduce turbulence within the germicidal radiation chamber 140.

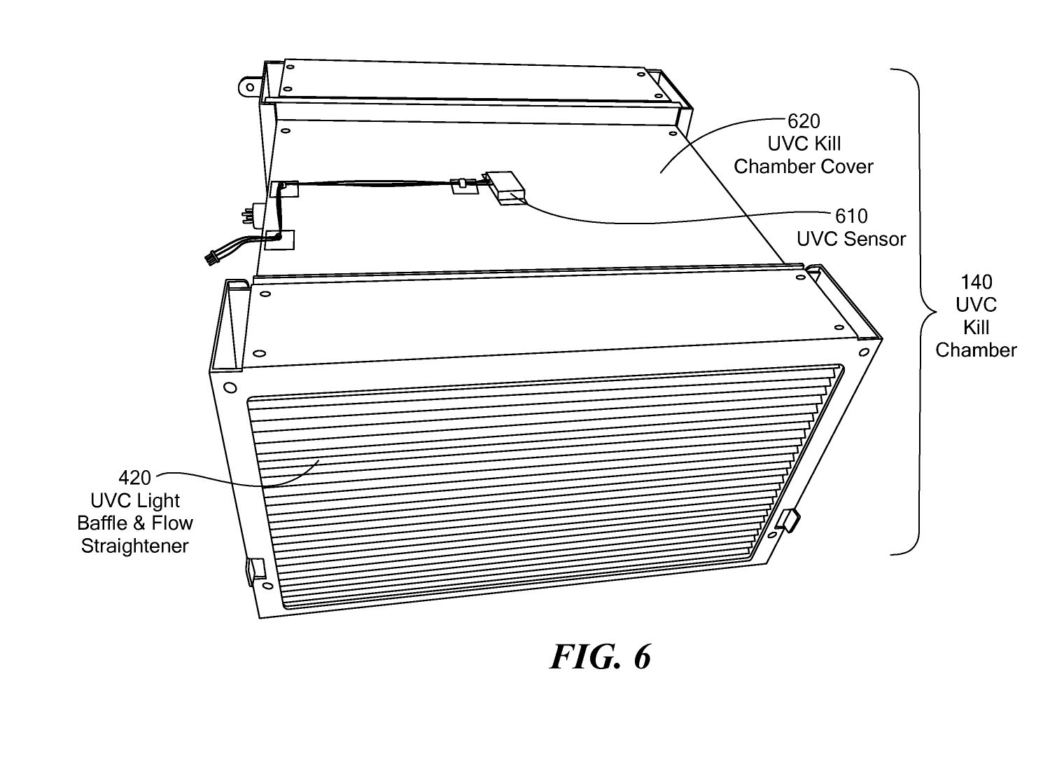

As shown in FIG. 6, the germicidal radiation chamber 140 can have a cover 620 that encases the germicidal radiation chamber 140. In addition, some embodiments of the present invention may also have a UV level sensor 610 located within the germicidal radiation chamber 140. The UV level sensor 610 can either be in or at the edge of the air flow. The UV level sensor 610 can transmit a signal to the microprocessor, which may control the fan speed or indicator lights based on the UV level sensor signal.

As shown in FIG. 7, the system 110 may also include an air flow sensor 710 located within the germicidal radiation chamber 140 (e.g., mounted to the inside wall of the chamber) and connected to the microprocessor. In preferred embodiments, the air flow sensor should 710 be a solid state sensor and co-linear with the air flow. In addition, the air sensor 710 should be shielded from the UV radiation to prevent damage to the air flow sensor 710. The air flow sensor 710 can send a signal to the microprocessor indicative of the air flow through the system. The microprocessor may then use this signal to modify the fan speed or control an indicator light (e.g., an alarm). In some embodiments, the air flow sensors 710 can be temperature compensated.

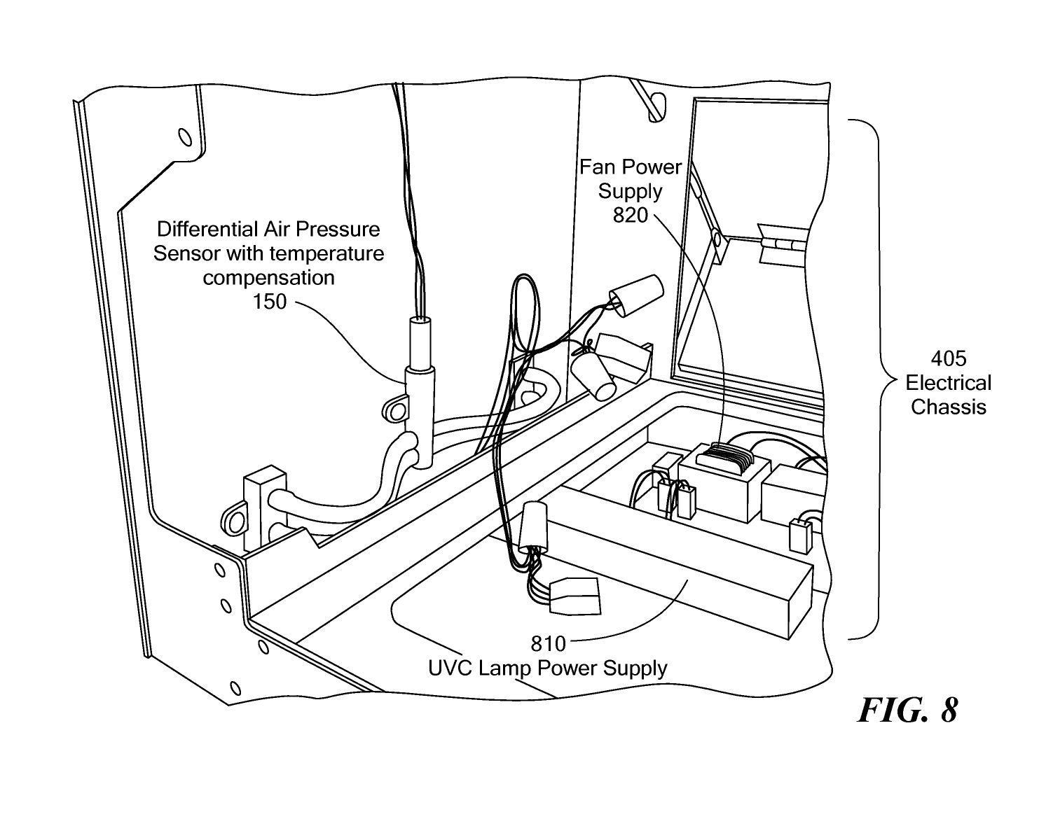

In addition to the above described components, the electrical chassis 405 can also house the UVC power supply 810 and the fan power supply 820 (FIG. 8). The electrical chassis 405 can also house the differential air pressure sensor 150. In a similar manner to the flow sensors 710, the differential air pressure sensor 150 can be temperature compensated.

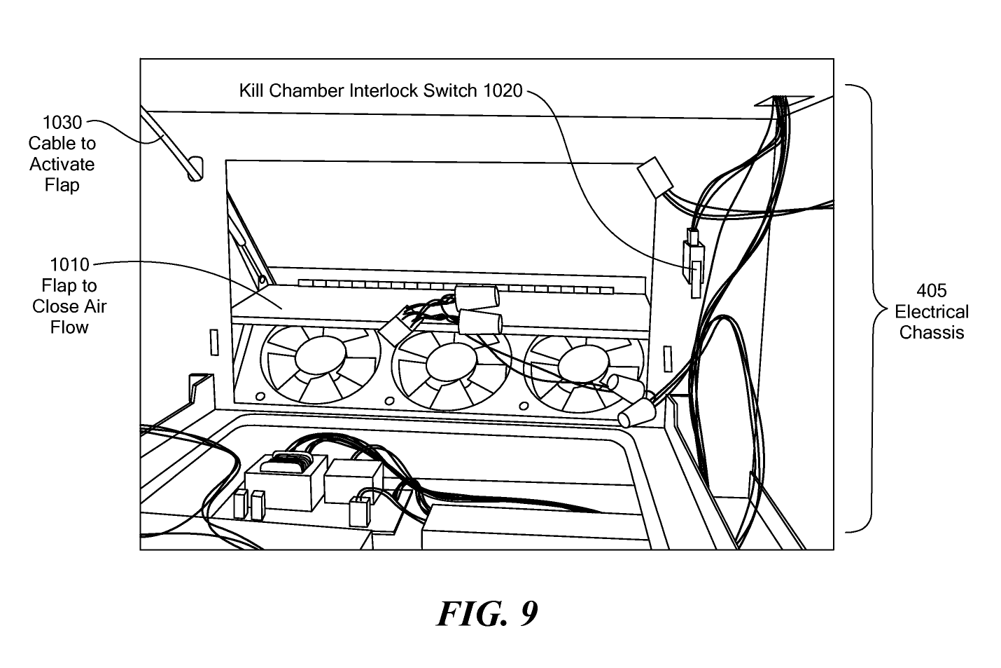

As shown in FIG. 9, to improve system storage and prevent debris, dirt, and other objects from collecting within the system 110, the system 110 may also have a cover 1010 that closes off the air flow when the system 110 is not in use. The cover 1010 may be, for example, a slide or a flap made from an insulating material. In some embodiments, the system may include a cover interlock switch 1020 electrically connected to the cover 1010 to sense the position of the cover 1010 (e.g., whether the cover is open or closed). The cover interlock switch 1020 may also be electrically connected to the microprocessor such that it prevents system operation when the cover 1010 is closed. For example, the interlock switch 1020 may send a signal to the microprocessor 360 indicating that the cover 1010 is closed.

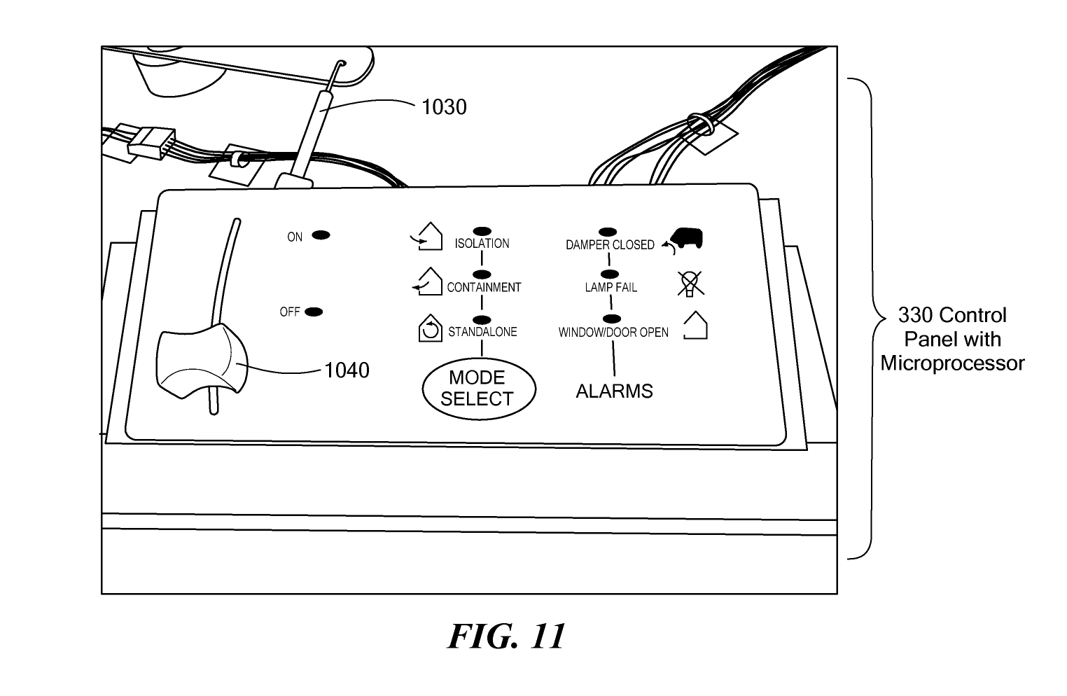

In some embodiments, a cable 1030 can be used to activate (e.g., open and close) the cover 1010. The position of the cable 1030 can act as the on-off switch for the system. For example, when the cable position corresponds to an open cover, the system 110 is on. Conversely, when the cable position corresponds to a closed cover, the system 110 is off. Like the cover 1010 itself, the cable 1030 can also be electrically connected to a cable interlock switch 1050 (FIG. 10) to sense the position of the cable 1030. A user can adjust the position of the cable 1030 (e.g., open and close) using a knob 1040 located on the system control panel 330 (FIG. 11).

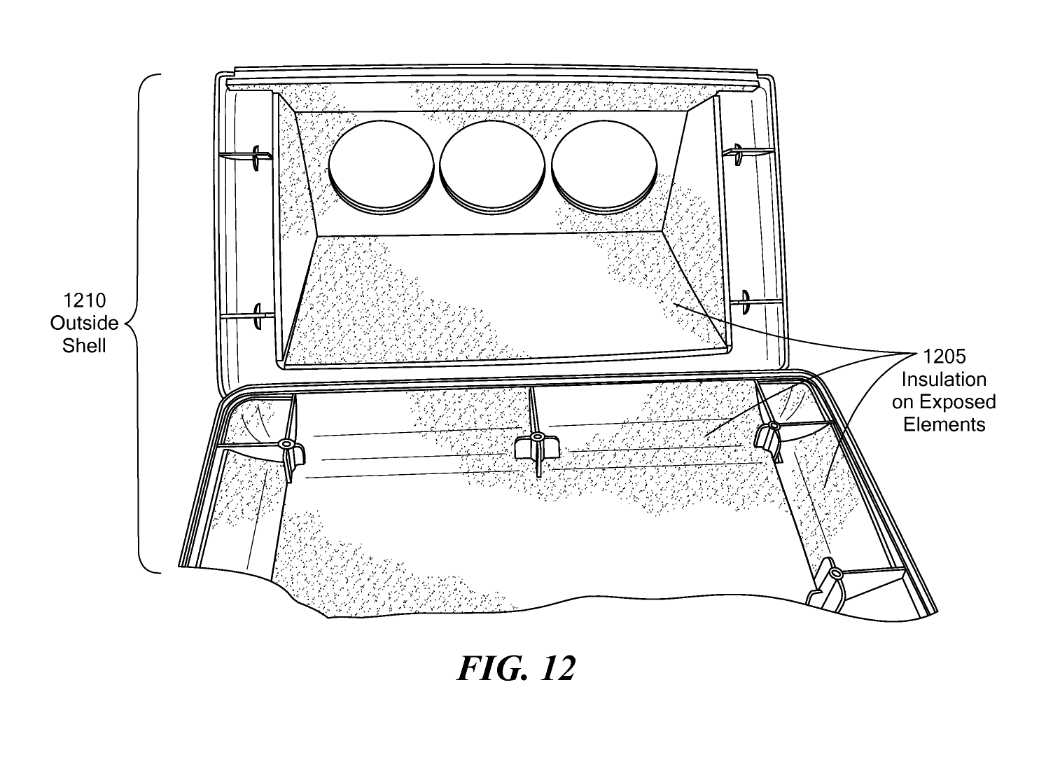

As shown in FIG. 10, the system can have a fan assembly 1025 attached to the electrical chassis 405. The fan assembly can have any number of fans (FIG. 10 shows 3 fans) that create the air flow through the system. As mentioned above, the fan speed can be controlled based on a number of criteria including, but not limited to, pressure differential, set points, and amount of UV light. The fan assembly 1025 can have a pre-filter assembly 1027 that covers each of the fans. The pre-filter assembly 1027 prevents larger objects, debris, or small animals from entering the system 110. In some embodiments, the portion of the system 110 exposed to the outside elements may have insulation 1205 (FIG. 12).

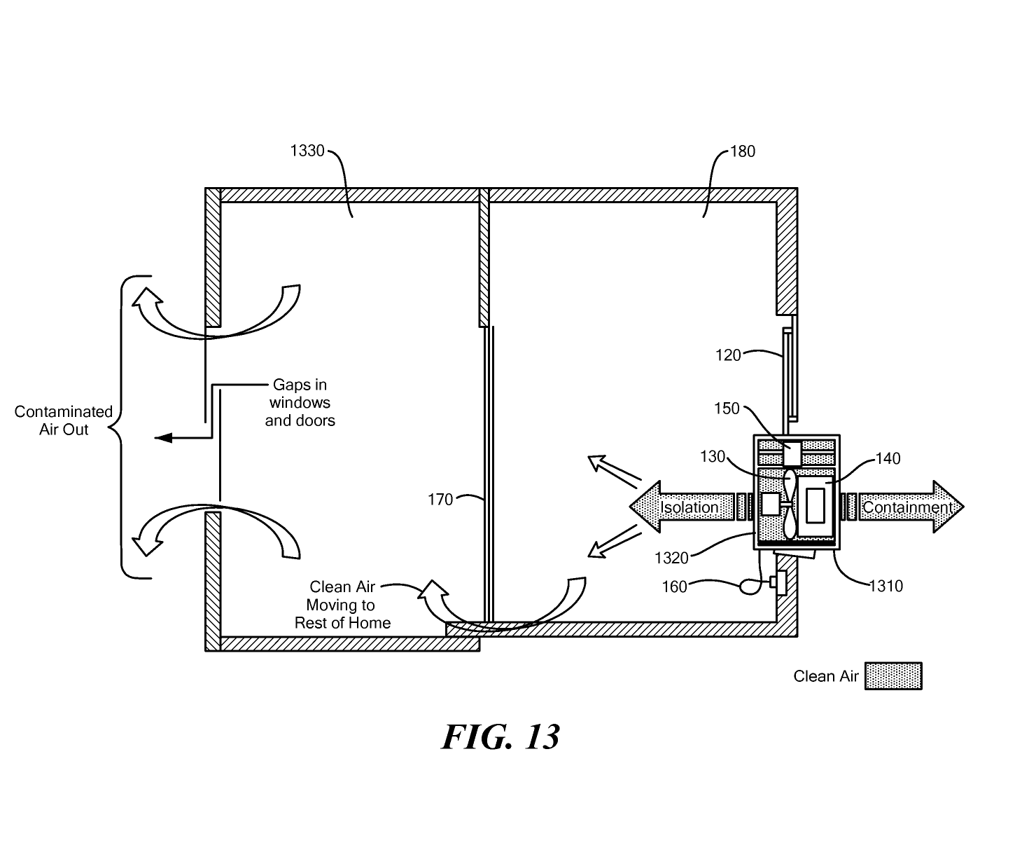

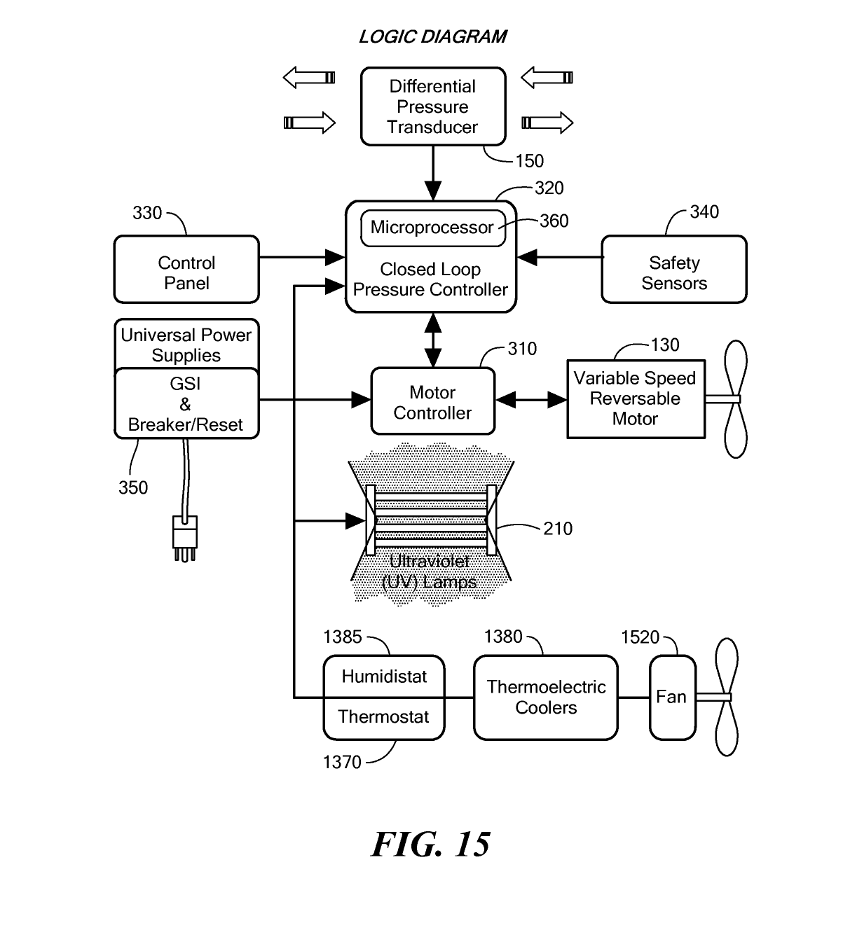

FIG. 13 shows an alternative embodiment of an air-pressure-isolation system 1310 in accordance with additional embodiments of the present invention. Like the system 110 shown in FIG. 1, the system 1310 shown within FIG. 13 may also be a through window, “plug and play” type system. As such, the system 1310 can similarly transform a closed space 180 into either an isolation or a containment room by placing the system 110 into a window 120 and plugging a power cord 160 into a standard wall socket. As shown in FIGS. 15-17 (discussed in greater detail below), the inward facing side 1320 of the system 1310 may have a stylish design so that it does not negatively impact the aesthetics of the closed space 180. The outward facing side of the system 1310 may have a design that is suitable for exposure to the environment.

In the isolation and/or containment configuration, the operation of the system 1310 shown in FIG. 13 is similar to that of the system 110 shown in FIG. 1. For example, in an isolation configuration, the variable speed fan(s) 130 force(s) clean air into the closed space 180, resulting in a positive pressure within the closed space 180. In order to produce a constant positive pressure consistent with surgical sites and clean rooms, the system 110 may control the air flow into the room, by varying the speed of the fan, to match the air flow out of the room through gaps around windows and doors. In the containment configuration, the variable-speed fan 130 forces air out of closed space 180, resulting in a negative room air pressure. In either orientation, a germicidal radiation chamber 140, located within a closed airflow path, cleans the air as it passes through system 110.

As discussed in greater detail below, the closed space 180 may be a room of a home, and the door 170 of the closed space 180 may lead to another room 1330 and/or the reminder of the home/building. In such instances, as also discussed in greater detail below, the system 1310 may be used to improve the air quality within the household.

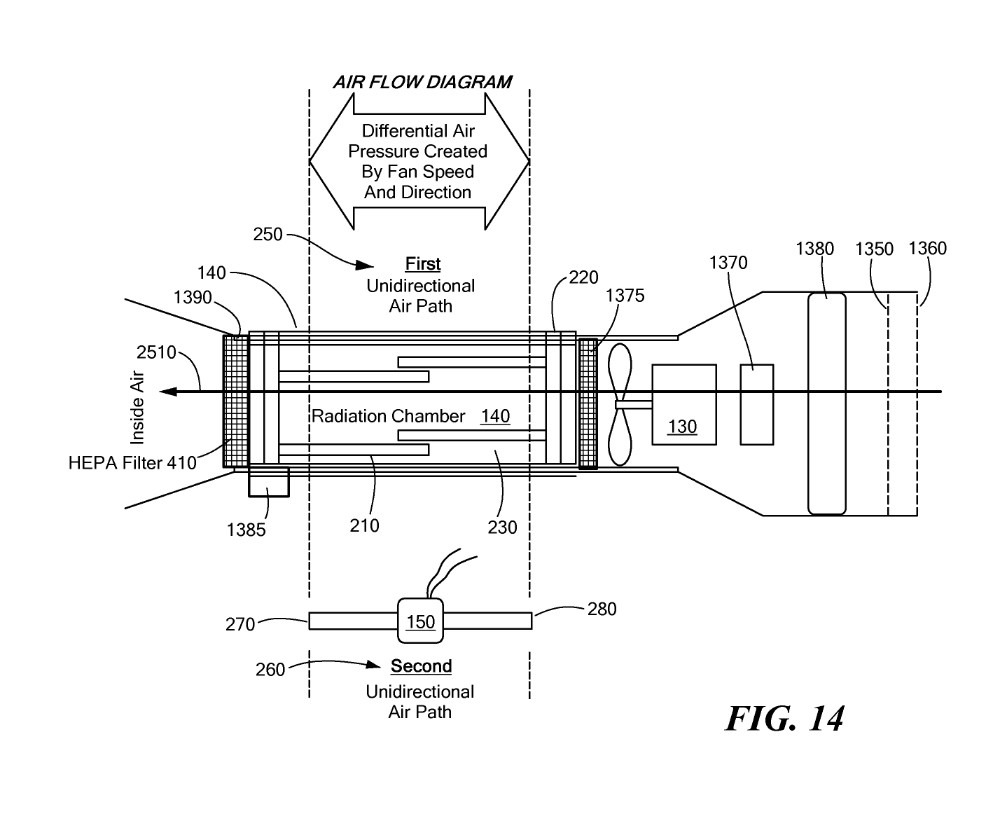

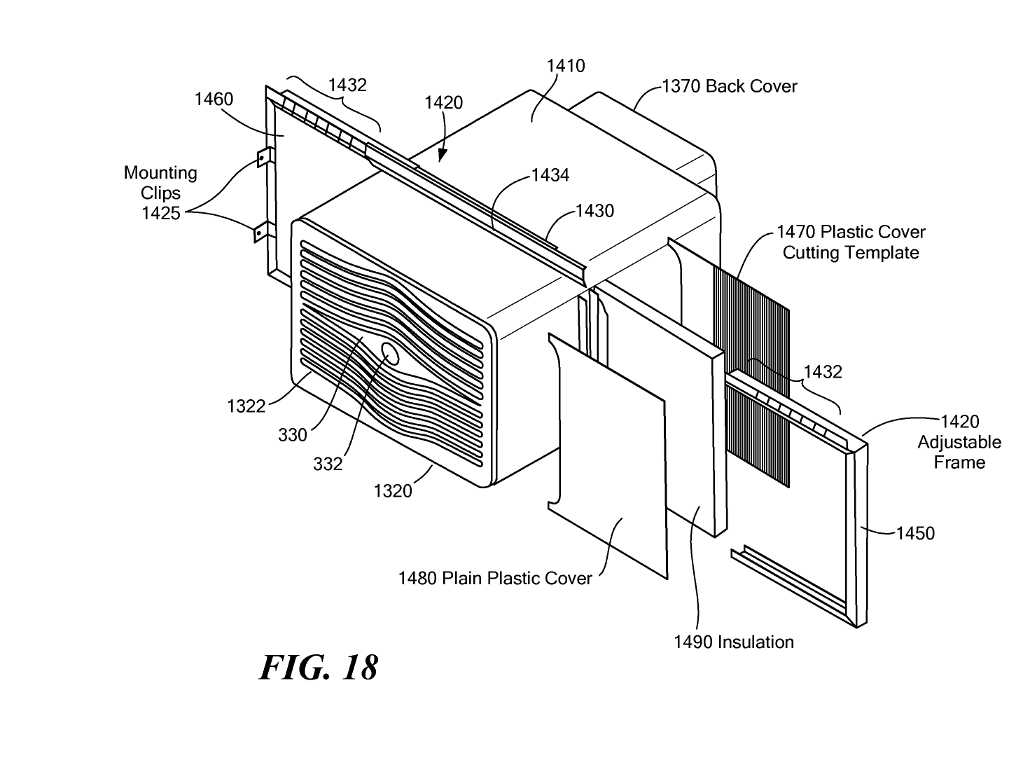

As show in FIG. 14, in addition to the germicidal radiation chamber 140 containing the ultraviolet lamps 210, the system 1310 may also include an intake plenum 1340 upstream of the fan 130. The intake plenum 1340 may include a pre-filter, a carbon filter 1350, and a bug screen 1360 to prevent debris, dirt, bugs, and other objects from collecting within/entering the system 1310. A back cover 1370 (FIG. 18) may be used to cover the components at the intake of the system 1310 (e.g., the back cover 1370 may be used to cover the intake plenum 1340). In the absence of the intake plenum 1340, the fan assembly 1025 can have a pre-filter assembly that covers the fan(s) (e.g., similar to that described above). The pre-filter assembly prevents larger objects, debris, or small animals from entering the system 1310.

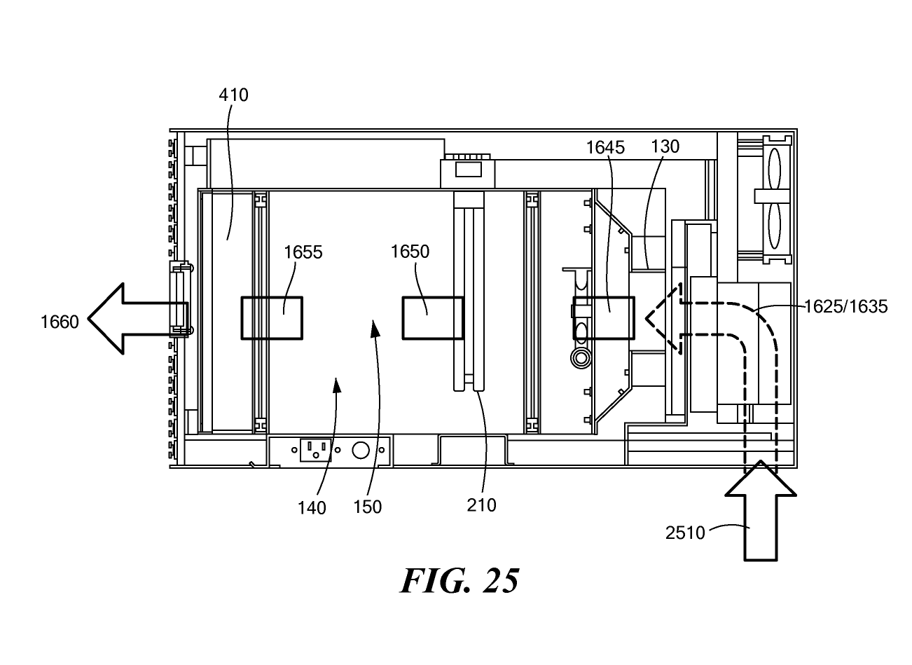

Additionally, in some embodiments, the system 1310 may have a thermostat 1370 located within the path of the external air 2510 (FIG. 25) drawn into system 1310. The thermostat 1370 may be connected to a heater/cooler 1380 (e.g., a reversible thermoelectric device). The thermoelectric device can pre-heat and/or pre-cool the air entering the system 1310 (e.g., the drawn air) based on a signal from the thermostat. For example, the thermoelectric device 1380 can heat and/or cool the drawn air based upon the temperature of the incoming air measured by the thermostat 1370 and/or the room temperature within space 180.

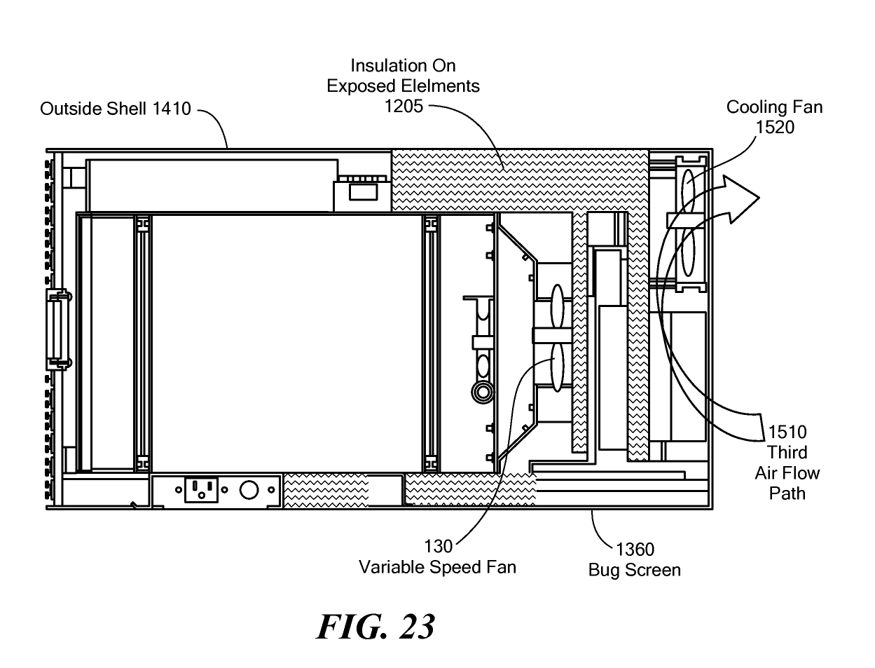

As shown in FIGS. 14 and 15, in embodiments containing the thermoelectric device 1380, the thermoelectric device 1380 may have one side exposed to the air flow 2510 and the other side exposed to a third air flow path 1510 (FIG. 23). The system 1310 may also include a second fan 1520 (e.g., a cooling fan) in the third air path 1510 (e.g., to draw air through the third air path 1510). The second fan 1520 may be activated when power is supplied to the thermoelectric device 1380.

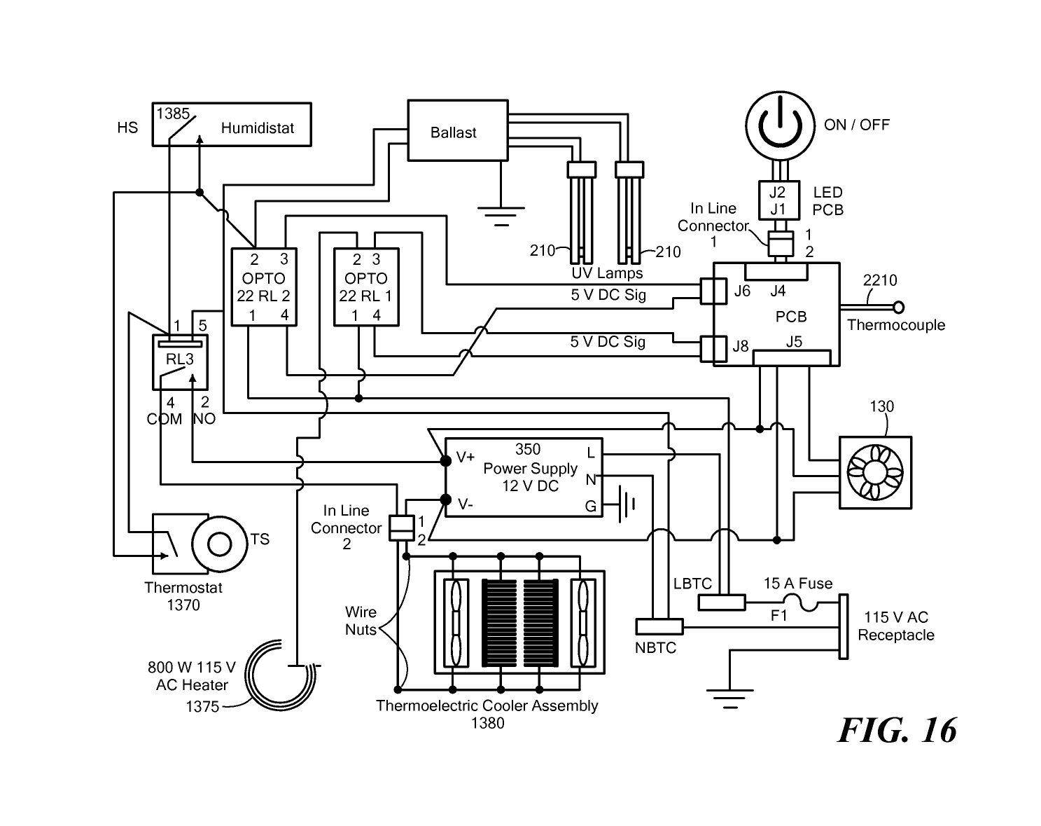

In order to sense/measure the humidity within the air flow path, some embodiments may also include a humidistat 1385 located in the air flow path (e.g., at the exit of the system 110). Like the thermostat 1370, the humidistat 1385 can also be connected to the thermoelectric device 1380 (FIGS. 15 and 16) to enable the thermoelectric device 1380 to cool (or pre-heat) and dehumidify the incoming air 2510. For example, based upon the temperature and humidity measurements, the thermostat 1370 and the humidistat 1385 can control the thermoelectric device 1380 to, in turn, control the temperature and humidity within the system 1310 and the temperature and humidity of the air exiting the system 1310 (e.g., into the space 180).

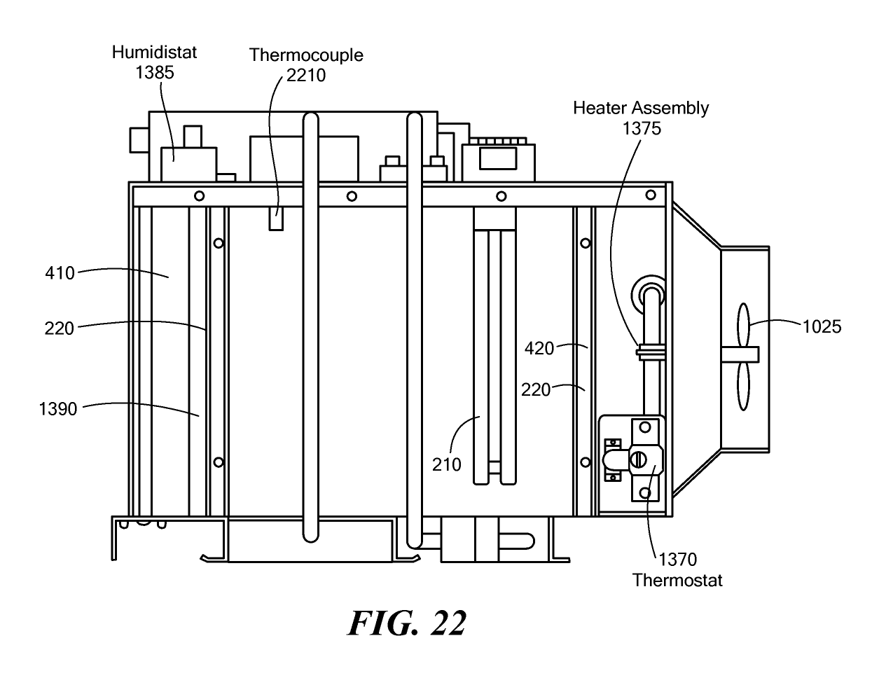

In addition to the humidistat 1385, thermostat 1370, and the thermoelectric device 1380, some embodiments may also have a main heater 1375 located just upstream of the radiation chamber 140, and a thermocouple 2210 (FIG. 22) located within the germicidal radiation chamber 140. The thermocouple 2210 may be connected to the closed-loop controller 320 which, in turn, can provide a modulated power to the main heater 1375. In this manner, in addition to dehumidifying and/or cooling the air passing through the system 1310, the system 1310 can also heat the air to prevent cold air (e.g., from outside of the home) from being introduced into the space 180/home 182.

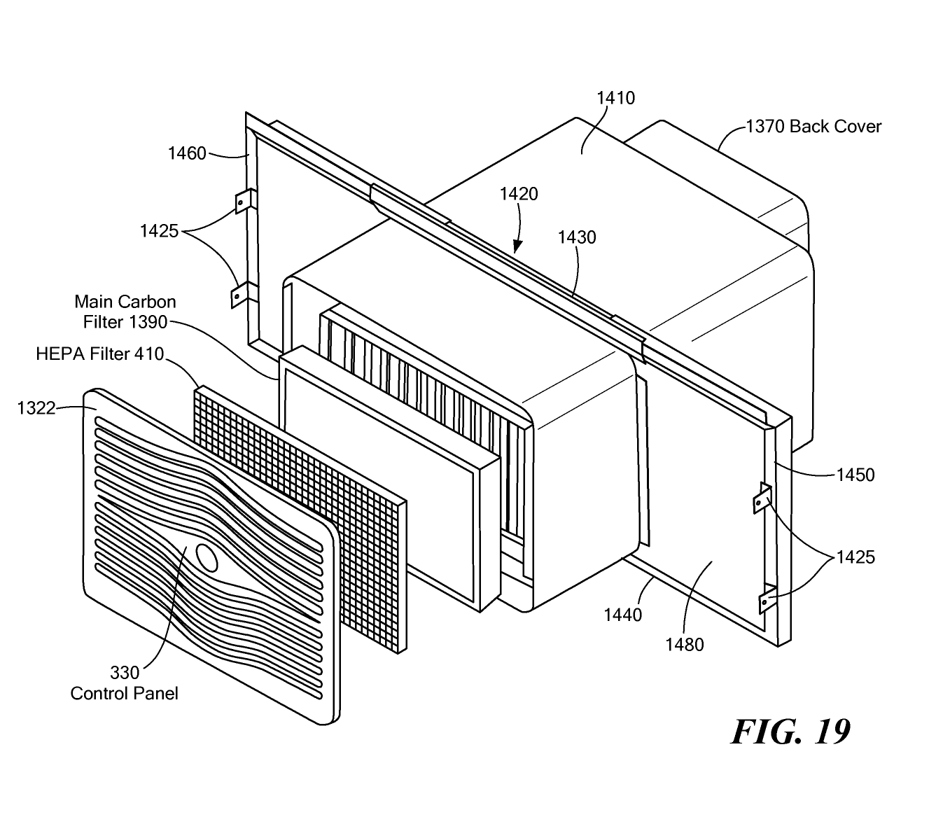

As discussed above, some embodiments of the present invention can include a HEPA filter 410 located at one or both ends of the germicidal radiation chamber 140. For example, as shown in FIGS. 14, 17, and 19, the HEPA filter 410 may be located at the outlet of the germicidal radiation chamber 140 (e.g., just behind the front cover 1322 and control panel 330). Additionally, upstream of the HEPA filter 410, some embodiments of the present invention can also include an additional carbon filter (e.g., main carbon filter 1390). As discussed in greater detail below, the filters (e.g., the HEPA filter 410, main carbon filter 1390, and pre-filter and carbon filter 1350) may be used to further clean and sterilize the air flowing through the system 1310.

Although the embodiments described above have control panels with a number of features (e.g., a switches, key pads, etc.), other embodiments can have a simpler control panel 330. For example, in true “plug and play” systems 1310, the control panel 330 can merely include an on/off button 332. As the name suggests, the on/off button 332 can be depressed by the user to turn the system 1310 off and on. All other control and operating conditions of the system 1310 (e.g., temperature, humidity, etc.) can be pre-programmed and automatically controlled by the system 1310. Additionally or alternatively, the control panel 330 can also include temperature and humidity controls (not shown) that allow the user to set a desired temperature and/or humidity of the air exiting the system 1310.

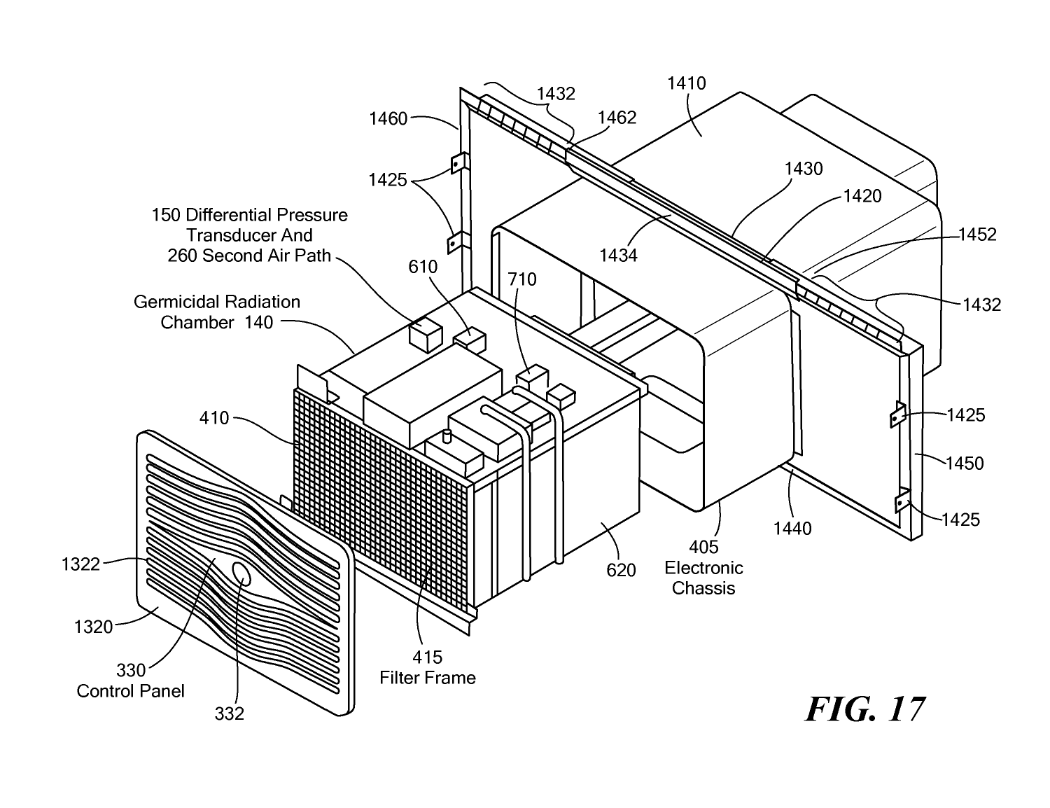

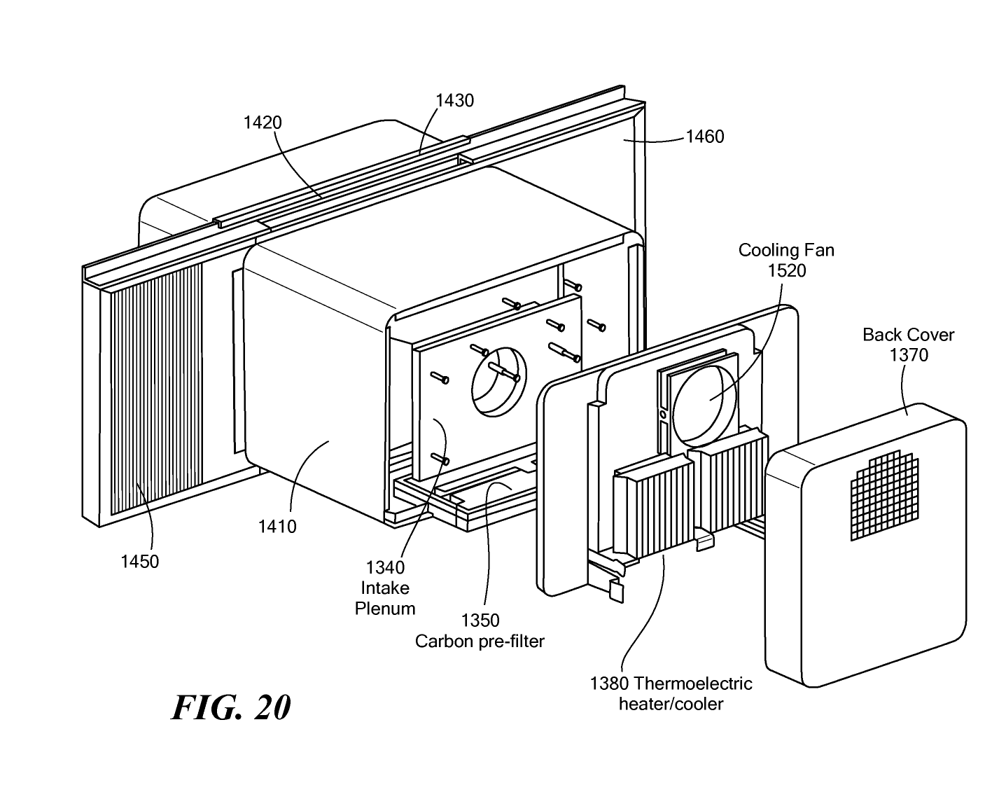

As shown in FIGS. 17-20, the system 1310 (or the system 110) can include an expandable frame 1420 extending around the periphery of the outside shell 1410 (or the electronic chassis 405). As mentioned above, various embodiments of the present invention can be configured for through window installation. To that end, the expandable frame 1420 can provide for a better fit in through-window installations. For example, the expandable frame 1420 can expand to the size of the window (e.g., the window frame) in which the system 1310 is installed. The expandable frame may include a soft gasket for sealing against the window sill, window frame, and the system shell.

As best shown in FIGS. 17-19, the frame 1420 can include a top rail 1430 extending across the top surface of the outside shell 1410, a bottom rail 1440 extending across the bottom surface of the shell 1410, and two adjustable side rails 1450/1460 that extend from the right and left sides of the shell 1410. The top rail 1430 of the frame 1420 can include a tape measure 1432 calibrated at half scale (½ inch to an inch) with the zero point 1434 at the center of the top rail 1430. The numbers may increase in both directions (e.g., to the left of the zero point 1434 and to the right of the zero point 1434).

Prior to installing the system 1310 into the window, the top rail 1430 and the two adjustable side rails 1450/1460 can be placed in an open window and expanded to fit the window frame. This, in turn, reveals the numbers on the measuring tape 1432. The top rail 1430 can then be moved so that the zero point 1434 is at the center of the window and the numbers at the ends 1452/1462 of the adjustable rails 1450/1460 match (e.g., so that the side rails 1450/1460 are equidistant from the shell 1410). The number showing at the ends 1452/1462 of the adjustable rails 1450/1460 can then be used to cut a plastic cover template 1470 (FIG. 18). The template 1470 can then be used to mark and cut plastic covers/panels 1480 and insulation 1490.

After cutting the cover/panels 1480 and insulation 1490, the individual installing the system 1310 can assemble the frame 1420 with the cut covers/panels 1480 and insulation 1490 located in the space between the side rails 1450/1460 and the sides of the shell 1410. The individual may then fasten the system 1310 to the window 120 using the mounting clips 1425. As mentioned above and as shown in FIG. 23, the portion of the system 1310 exposed to the outside elements may have insulation 1205.

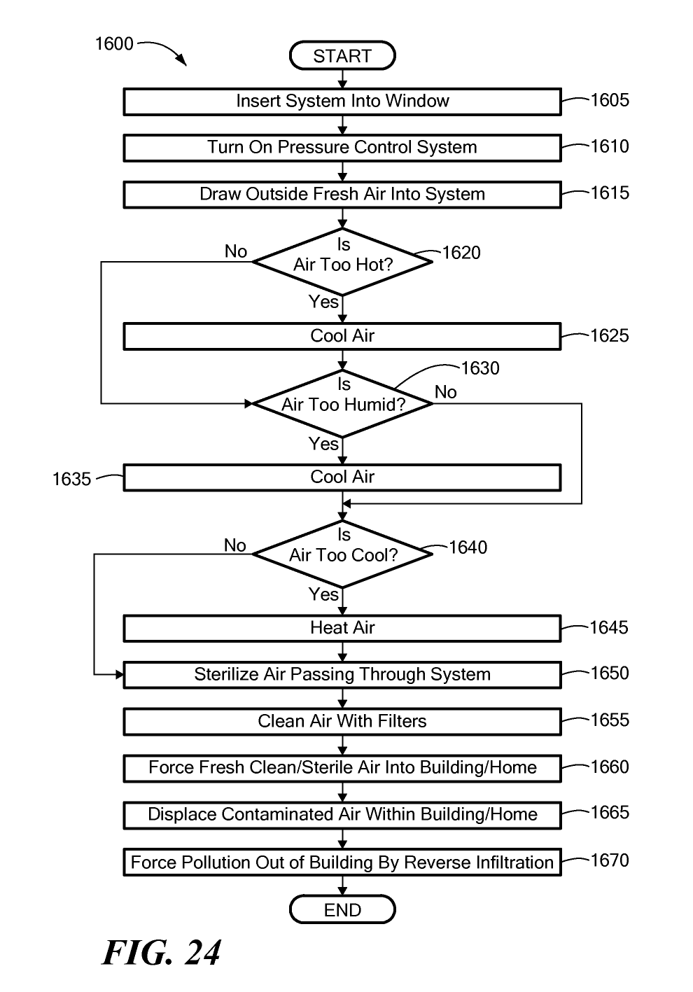

In addition to being used for creating the isolation and/or containment rooms discussed above, some embodiments of the pressure control system 1310 (and/or system 110) can also be used to improve the air quality within a household. For example, the control system 1310 may be placed within the window 120 of a room in a house 182, and can be used to replace contaminated air within the home with clean/sterile air. FIG. 24 shows one embodiment of a method of air replacement in accordance with the present invention. The location of the drawn air within the system 1310 during the various stages of the method discussed below is shown in FIG. 25.

According to the method 1600, the homeowner (or other individual), can install the air-pressure control system 1310 into a window of the home (Step 1605), and turn on the system (Step 1610). It is important to note that, instead of a window, the air-pressure control system 1310 may be installed into a doorway, or other opening within the home that allows the system 1310 to draw in air from outside of the home (e.g., any opening that passes through an exterior wall of the home).

Once installed into the window and turned on, the system 1310 will draw in air from the exterior of the home (Step 1615). In some embodiments, the air drawn from the exterior of the home can be conditioned to room temperature and humidity (e.g., the temperature and humidity within the home). For example, if the system 1310 determines that the temperature is above room temperature (e.g., using the thermocouple 2210) (Step 1620) or that the incoming air is too humid (e.g., using the humidistat 1385) (Step 1630), the system 1310 can cool the air (Steps 1625 and 1635) using the coolers within the thermoelectric device 1380. Conversely, if the system 1310 (e.g., the thermocouple 1385 and/or thermostat 1370) determines that the incoming air is too cold (Step 1640), the system 1310 activate the main heater 1375 to heat the drawn-in air to room temperature (Step 1645).

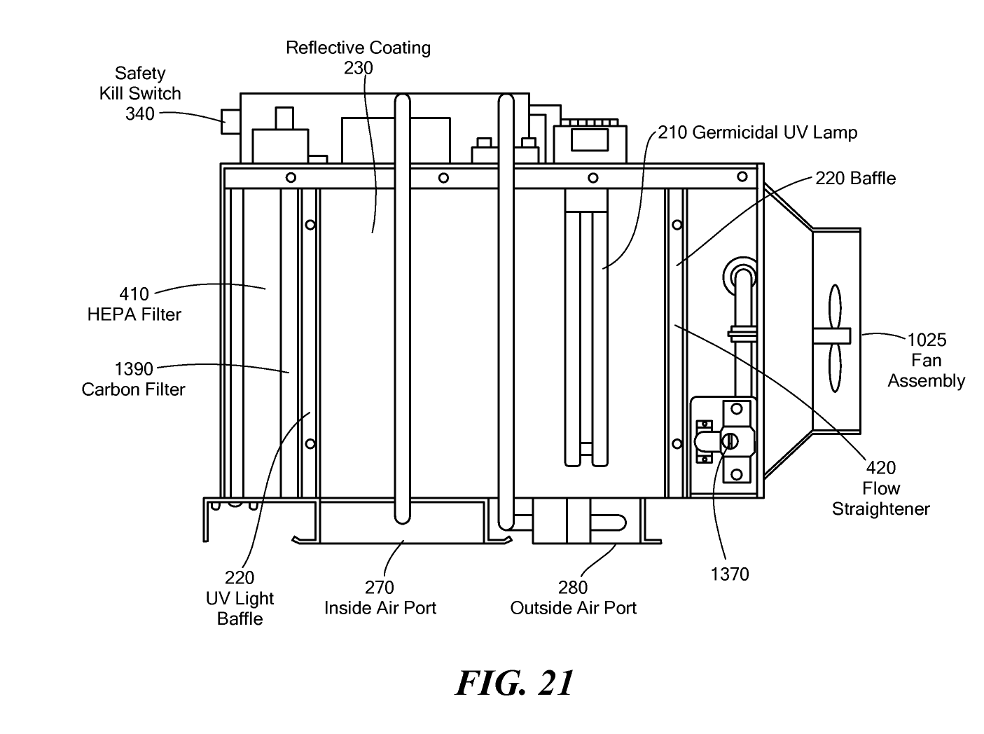

As mentioned above, some embodiments of the system 1310 can have a germicidal radiation chamber 140 and/or one or more filters (e.g., the HEPA filter 410, the main carbon filter 1390, and/or the pre-filter and carbon filter 1350) located on either side of the germicidal radiation chamber 140 (FIGS. 14, 17, 19 and 21). To that end, some embodiments of the control system 1310 can sterilize (Step 1650) (e.g., in those embodiments that include the germicidal radiation chamber 140), and clean (Step 1655) (e.g., in those embodiments that include the filter(s)) the air as it passes through the airflow path and the germicidal radiation chamber 140. For example, as the drawn air passes through the germicidal radiation chamber 140, the UV light can destroy bacteria and micro-organisms (e.g., viruses, bacteria, fungus, mild, and spores) within the air. Additionally, the filter(s) 410 can remove/trap micro contamination within the airflow.

The cleaned and/or sterilized air may then be introduced into the home (e.g., into the room/space 180 in which the air-pressure control system 1310 is located) (Step 1660). As the cleaned/sterilized air is introduced into the room, an equal volume of contaminated air within the room/home is displaced (e.g., for each cubic foot of air that is introduced into the room/home, a cubic foot of air is displaced out of the room/home) (Step 1665). The air replacement will begin at the entrance point of the cleaned/sterile air (e.g., at the outlet of the air-pressure control system 110) and will gradually move throughout the room into the adjoining room 1330 and the remainder of the home 182. As additional contaminated air is displaced and replaced by clean/sterile air, the airborne contamination throughout the entire living space is forced out of the building/home 182 (Step 1670) (e.g., by reverse infiltration), and the overall contamination level within the building/home 182 is reduced.

It is important to note that the germicidal radiation chamber 140 and the filter(s) can, together, remove substantially all of the contamination within the air drawn from outside of the home. For example, the output of the air-pressure control system 1310 (e.g., the air introduced into the room/home) can contain substantially no particles ranging in size from 5.0 microns to 0.3 microns. Therefore, as the contaminated air is displaced and replaced with the air being output by the air-pressure control system 1310, the air quality within the home improves, and any harmful particulates and/or volatile organic compounds (VOCs) (e.g., any airborne contamination) are removed from the home.

As mentioned above, the air-pressure control system 1310 can control the speed of the fan 130 about a set point using the microprocessor 360 and/or closed-loop controller 320. The set point can be preset at the factory during manufacturing or the set-point can be input into the system by the end user (e.g., before or just after inserting the control system 1310 into the window). For example, the set-point can be preset (or set by the end user) to control the fan to maintain an airflow rate of between 24 cubic feet per minute (CFM) and 75 cubic feet per minute (CFM). The airflow rate set points of between 24 CFM and 75 CFM are merely examples, and the airflow rate can be set to any suitable flow rate. In some embodiments, the flow rate can be dependent upon the size of the room/home, the level of contamination within the home, the time desired to clean the room and/or home, and/or the expected level of contamination of the air outside the home (e.g., the air being drawn into the system 1310).

### Exemplary Study:

Using an air-pressure control system in accordance with various embodiments of the present invention, a study was conducted to explore the ability of some embodiments of the present invention to reduce airborne contamination within the home by replacing contaminated air within the home with clean air (e.g., Air Replacement Technology (A.R.T.™)). The study compares A.R.T. to a recent national study that used air filtration products to reduce airborne contamination and quantify the health benefits. The pilot study was conducted in seven homes identified by the Massachusetts Support Group of the Alpha 1 Association.

The study and the Air Replacement Technology (A.R.T.) is based on two scientific principles—(1) that two bodies cannot occupy the same space at the same time, and (2) the effects of differential air pressure creating air flow. Based upon the above, it was determined that, for each cubic foot of clean, sterile air pushed into the home, a cubic foot of contaminated air is forced out. Each cubic foot of air leaving the home will contain contamination which will include mixtures of particulate and gases (triggers).

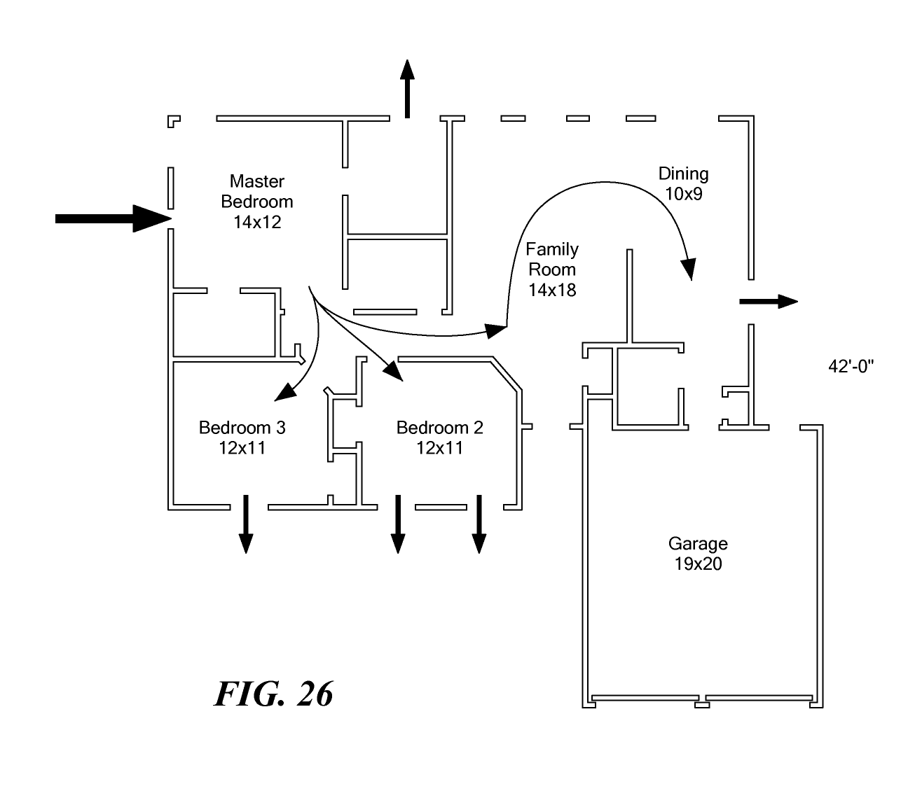

The process of air replacement technology begins at the entrance point of fresh sterile air (e.g., at the exit of the air-pressure control system 110/1310) and gradually moves throughout the home, eventually reducing airborne contamination throughout the entire living space (FIG. 26). It is important to note that, in contrast to air replacement technology, traditional re-circulating air filter products are designed to address particulate contamination and do not address the issue of introducing clean, fresh air or removing volatile organic compounds (VOC's).

The objective of the study was to determine the effectiveness of air replacement technology in a typical home setting. Air-pressure control systems 110/1310 were installed in the homes of seven members of the Massachusetts Support Group of the Alpha 1 Association. The homes varied in style, size and occupancy, with some including pets. The homes were constructed between 1960 and 2000 and heated with forced hot water or forced hot air. A requirement of the study was the availability of a double hung window to accommodate the system installation. The majority of the installations occurred in late summer to include both the fall allergy season and part of the winter heating season,

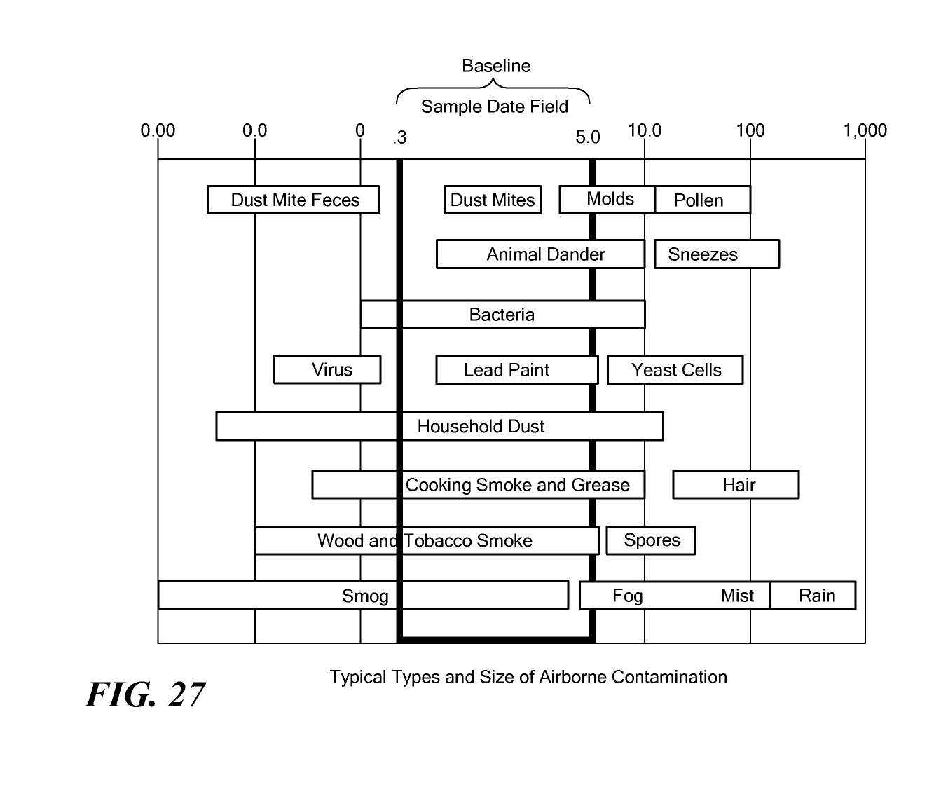

Once installed, the computer/controller of each of the installed systems controlled the differential pressure to create a stream of conditioned, clean, fresh, sterile air into the home, which, in turn, displaced all sizes and types of airborne micro-contamination, VOC's, and harmful gases. FIG. 27 shows a distribution of harmful pathogens. The airborne particulate levels were monitored in the size range typical of pathogens responsible for respiratory exacerbation. Baseline indoor air pollution data was collected at the time of installation of the air-pressure control system and measured monthly in each home for an average of four months.

All particle count data was taken with a MetOne GT-321 Hand Held Particle Counter. The data taken at installation and throughout the study includes five different particle sizes from 5.0μ to 0.3μ. All site visits verified that the clean, fresh sterile air entering the room from the air-pressure control system contained zero particles from 5.0μ to 0.3μ. Particle counts were also taken at the center of the room in which the air-pressure control system was installed, and in a kitchen or living room chosen by the participant.

The data presented below focuses on the most dangerous particle size (0.30μ), and all calculated averages are based on concentrations of 0.3μ particles. During installation data was taken in all homes at both the first and second location. The data was then averaged to determine a baseline concentration of 1,231,493 at 0.3μ particles per cubic foot at the first location, and 858,516 at 0.3μ particles per cubic foot at the second location. At each subsequent visit, the data from each location was averaged and compared to the baseline data for those locations and reported as a percent of particulate reduction. Table 1 shows the percent particle reduction at location one and Table 2 shows the percent particle reduction at location two.

TABLE 1
Percent Particle Reduction at Location #1

| | At Time | | | | | Average | Average |
|---|---|---|---|---|---|---|---|
| | of Install | Average | Average | Average | Average | Particle | Percent (%) |
| | Average of 7 | Reading | Reading | Reading | Reading | Count for | Particle |
| | Homes | Visit #1 | Visit #2 | Visit #3 | Visit #4 | visits 2-7 | Reduction |
| Particle | Zero | Zero | Zero | Zero | Zero | Zero | Zero |
| Count @ | | | | | | | |
| System | | | | | | | |
| Particle | 1,231,493 | 383,206 | 304,610 | 291,030 | 519,309 | 374,539 | 70% |
| Count in | | | | | | | |
| Room | | | | | | | |
| Cleaned by | | | | | | | |
| System | | | | | | | |

TABLE 2
Percent Particle Reduction at Location #2

| | | | | | | | Average |
|---|---|---|---|---|---|---|---|
| | | Average | Average | Average | Average | Average | Percent (%) |
| | At Time | Reading | Reading | Reading | Reading | Particle Count | Particle |
| | of Install | Visit #1 | Visit #2 | Visit #3 | Visit #4 | for visits 2-7 | Reduction |
| Particle | 858,516 | 477,687 | 487,683 | 469,231 | 239,297 | 418,474 | 51.3% |
| Count in | | | | | | | |
| Additional | | | | | | | |
| Room | | | | | | | |

It is important to note that the choice of a cubic foot of air as a sample size has respiratory significance. In particular, the average adult inhales about one cubic foot of air per minute. The concentrations of dangerous 0.3μ, particles tracked in this study have respiratory significance because (1) they float and stay airborne for days, (2) the 0.3μ size particles can travel deep into the lungs, and (3) they can be absorbed by the body and trigger respiratory inflammation.

The above data shows that air replacement technology provided the greatest improvement in indoor air quality at the point of installation. Throughout the study, the particle count of the replacement air delivered by the air-pressure control system was zero for particles between 5.0 and 0.3 microns. The zero particle count readings at the air-pressure control system were consistent for all homes for the duration of the study.

The effectiveness of the fresh sterile air being introduced into the air-pressure control system installed location varied with the greatest individual reduction in airborne particulate of 87% and an average 4 month group reduction of 70%. (See Table 1)

As mentioned above, data was also taken in all homes at a second remote location. The contribution of the supply of fresh sterile air flowing through the first location to the second location also varied. The greatest individual reduction in airborne particulate was 77% with an average 4 month group reduction of 51%. (See Table 2)

Two short studies were also conducted to determine how quickly a room responds to air replacement technology. In both cases, the room responded at a contamination reduction rate of approximately 1% per minute.

### CONCLUSIONS

The use of available air cleaners to determine the health benefits of reducing airborne particulate in the home was previously reported in a 2011 nationwide study funded by National Institutes of Health (NIH). The NIH study found that a 20% reduction of airborne particulate results in an 18% reduction of unscheduled hospital visits. It is important to note that the NIH study preceded the introduction of air replacement technology (A.R.T.) and the air-pressure control systems described herein. The NIH was constrained by the use of available air cleaning technology study, and specifically expressed disappointment in removing only 20% of the airborne particulates, leaving all other forms of airborne pathogens behind.

In sharp contrast, various embodiments of the present invention removed 70% of the particulates. Furthermore, based on the physics of particle disbursement, all forms of indoor air pollution were present in each cubic foot of air that left the home.

The data taken at the second location supports the concept of differential air pressure transporting the benefits of the clean fresh air to other parts of the home. The transport of clean, fresh air to other parts of the home is a vast improvement over the localized air cleaning limitations of re-circulating air filter cleaners.

FIG. 28 shows an alternative embodiment of an air purification system 2800. In addition to many of the operational features and components discussed above (e.g., fans 130/910, power supply 350, motor controller 310, closed-loop controller 320, sensors 340/610/710, microprocessor 360, filter(s) 410/1027/1350/1390, heater/cooler 1380, etc.), the system 2800 can include a removable front bezel 2810 that connects to the body 2820 (e.g., the housing) of the system 2800 and allows the user to easily gain access to the internal components. The bezel 2810 may include a magnet 2815 that may secure the front bezel 2810 to the rest of the system 2800. Alternatively, the bezel 2810 may be secured to the rest of the system 2800 by any other means that allows a user to easily remove the bezel 2810 (e.g., by hand and without tools).

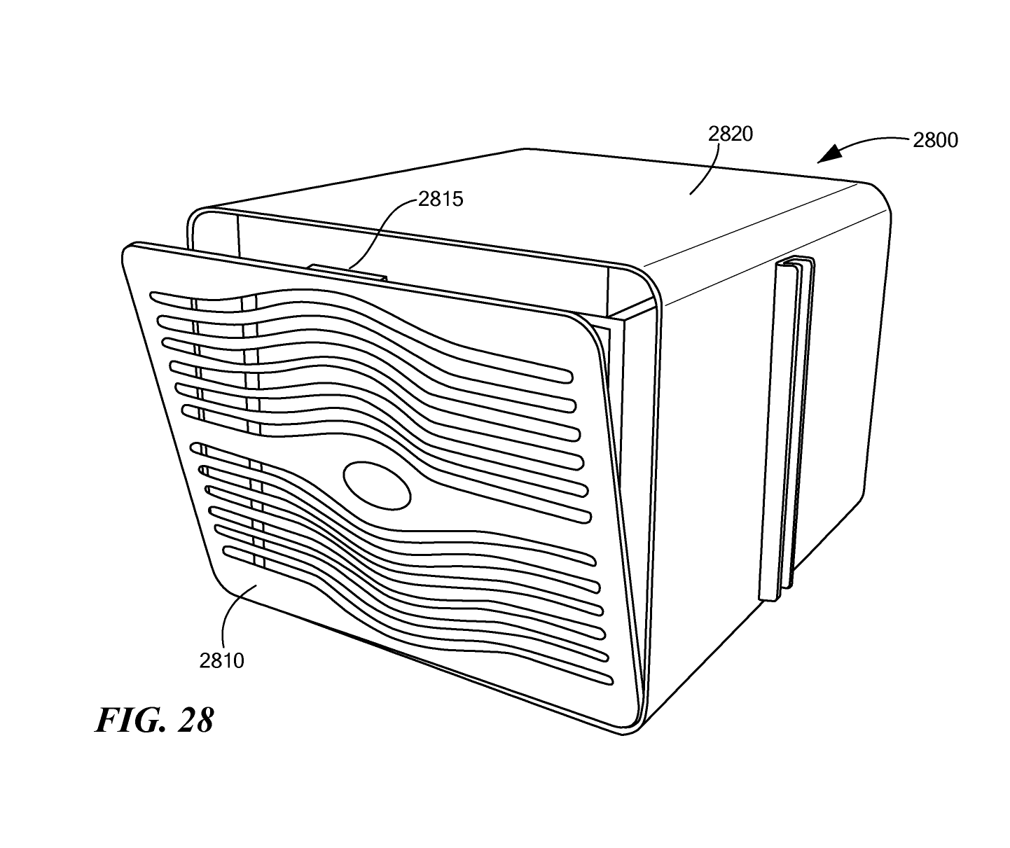

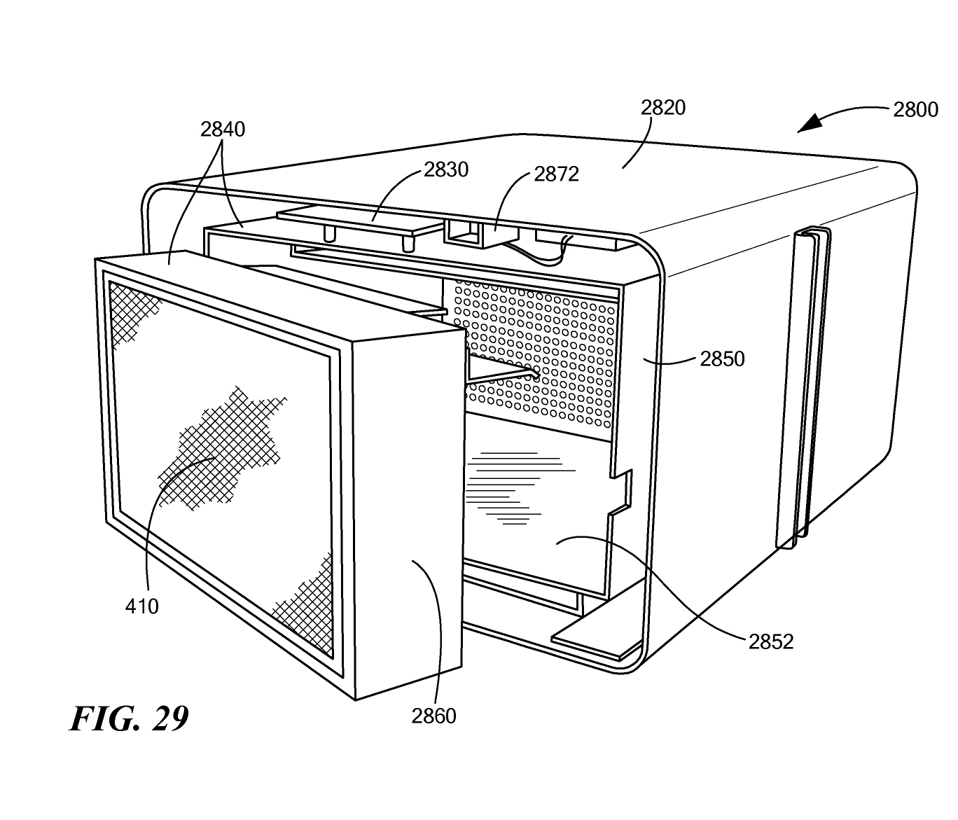

In addition to securing the bezel 2810 to the body 2820, the magnet 2815 may also be used in conjunction with a hall effect transistor 2830 to act as a safety on/off switch. For example, as shown in FIG. 29, the system electronics can include a hall-effect transistor 2830 located toward the front of the system 2800 (e.g., near the front bezel 2810 and magnet 2815). When the bezel 2810 is secured on the system 2800, the magnetic field created by the magnet 2815 will energize the hall effect transistor 2830, allowing the system 2800 operate. Conversely, when the front bezel 2810 is removed, the magnetic field will similarly be removed (e.g., because the magnet 2815 is on the bezel 2810). This, in turn, will de-energize the hall effect transistor 2830 and will cause the system 2800 to turn off. In this manner, the hall effect sensor 2830 acts as a safety switch that shuts off the system 2800 in the event that the front bezel 2810 is removed and the internal components are exposed.

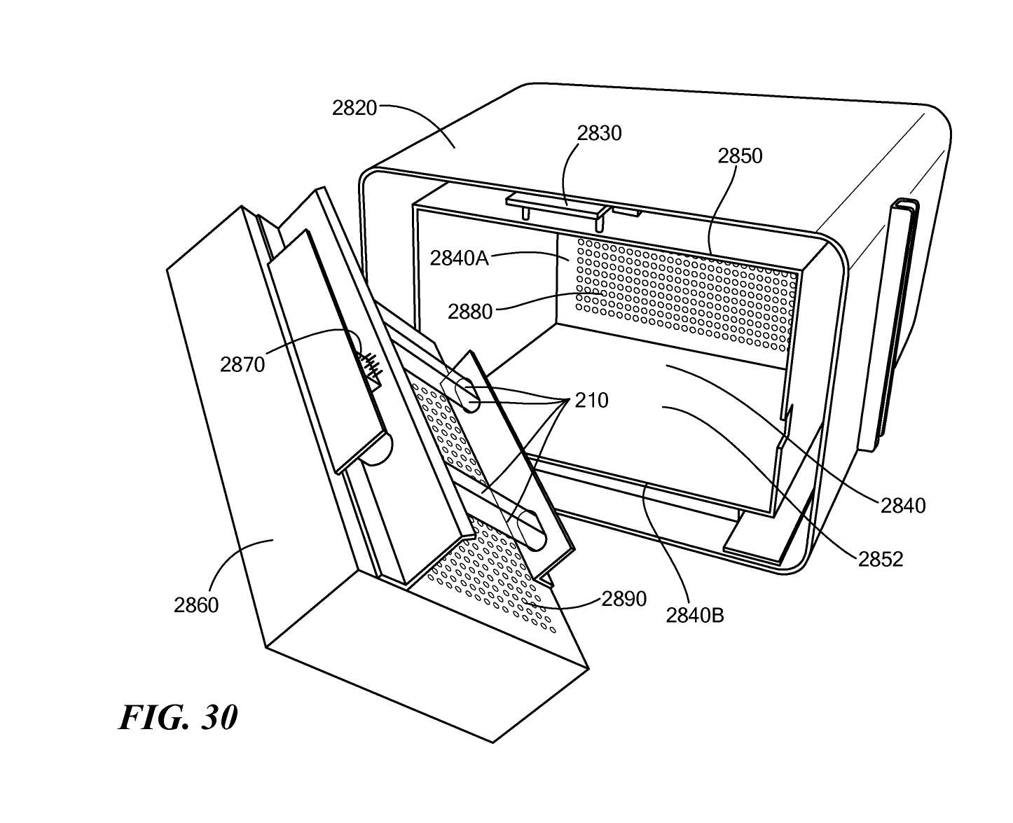

As shown in FIGS. 29 and 30, the germicidal radiation chamber 2840 may be formed by an electrical chassis 2850 and a removable cartridge 2860. The electrical chassis 2850 is secured within the body 2820 of the system 2800 and houses may of the electrical and mechanical components of the system 2800 (e.g., in a manner similar to the electrical chassis 405 discussed above). The chassis 2850 may have a hollow interior that forms the main chamber 2852 of the germicidal radiation chamber (e.g., the chamber through which air flows and in which the UV light from the lamps 210 begins to sterilize the air).

As best shown in FIG. 30 (which shows the cartridge 2860 removed), the cartridge 2860 contains many of the disposable components of the system (e.g., those items that may need to be replaced from time to time). For example, the cartridge 2860 may contain a number of filters (e.g., any of those filters discussed above including but not limited to the HEPA filter 410). To provide the cartridge 2860 with power (e.g., to operate the UV lamps 210), the cartridge 2860 includes an electrical plug 2870 that plugs into a corresponding plug 2872 (or receptacle; FIG. 29) on the chassis 2850 (e.g., the rest of the germicidal radiation chamber 2840). This creates an electrical connection with the chassis 2850 and provides power to the UV lamp(s) 210 within the cartridge 2860.

As mentioned above, the cartridge 2860 may be removable. To that end, when one of the components of the cartridge 2860 (e.g., a UV lamp or a filter) needs to be replaced, the user can simply remove the front bezel 2810 (from inside of the building) of the system to gain access to the cartridge 2860. AS discussed above, removal of the bezel 2810 will de-energize the hall effect transistor 2830 and stop the system 2800, if running. The user may then disconnect the cartridge 2860 from the chassis (e.g., by simply pulling on the cartridge 2860). Once the cartridge 2860 is disconnected, the user may replace the faulty or worn out component(s) and reinstall the same cartridge 2860 (e.g., the cartridge may be reusable) or simply install an entirely new cartridge 2860.

Additionally, as best shown in FIG. 30, like the embodiments discussed above, the system 2800 can include baffles and/or flow straighteners 2880/2890 located at either end of the system 2800. For example, the system 2800 may include a first baffle 2880 and/or flow straightener 2890 located at the inlet end 2840A of the germicidal radiation chamber 2840 and a second baffle 2880 and/or flow straightener 2890 located at the outlet end 2840B of the germicidal radiation chamber 2840. In some embodiments, the baffle 2880 and/or flow straightener 2890 at the outlet end 2840B may be part of the removable cartridge 2860. The baffles and/or flow straighteners 2880/2890 help prevent UV light within the chamber 2840 from escaping, but may be perforated to allow air to flow through the baffles and/or flow straighteners 2880/2890 and, therefore, through the chamber 2840 and system 2800.

One or both of the baffles and/or flow straighteners 2880/2890 can include a Titanium Oxide (TiO₂) coating (e.g., the baffle(s) 2880 can be plated with TiO₂). By coating the baffles 2880 (or the flow straighteners 2890) with TiO₂, some embodiments are able to utilize a photo catalytic process to help clean/purify the air passing through the system 2800. For example, the TiO₂ coating acts as a catalyst to covert ambient water vapor, ozone, and VOCs into less harmful components when exposed to the UV light from the lamps 210.

Unlike some of the embodiments discussed above that provide a variable flow of fresh cleaned air into a building and maintain a constant differential air pressure between the inside and outside of the building, other embodiments of the present invention utilize a constant displacement strategy. In such embodiments, the system (e.g., system 2800) is operated at peak air flow and peak efficiency at all times and lets the positive air pressure vary according to the porosity of the structure/building. For example, when the structures/buildings are well sealed, the air pressure will rise until the air expelled equals the set point of the incoming air flow rate (e.g., the setpoint of the system 2800). This assures that the time required to displace the contaminated air is minimized. Additionally, the constant displacement strategy allows the air flow rate (e.g., the maximum mass flow) to be set at the highest flow possible while still maintaining the max filtration efficiency and max UV sterilization.

The highest air flow rate (e.g., the set point) may be determined based on normal outdoor ambient weather (approximately 70° F. and 55% relative humidity). Alternatively, the system 2860 may be slowed (e.g., the highest air flow rate/set point may be reduced) to reduce the thermal load if the ambient weather conditions are hot (e.g., significantly above 70° F.), cold (significantly below 70° F.), and/or humid (significantly above 55% relative humidity). For example, the microprocessor 310 may monitor the temperature and relative humidity of the air entering the system (e.g., using the humidistat 1385 and thermocouple 2210), and adjust the set point/system operation accordingly. Adjusting the set point in this manner helps optimize system operation and increase the longevity of the system.

It is important to note that embodiments utilizing the constant displacement strategy discussed above may utilize a somewhat different control system and, in some instances, the air purification system 2800 may be simplified. For example, embodiments utilizing the constant displacement strategy do not require a pressure transducer and the second air path through the system, discussed above. However, in a manner similar to that described above, the system 2800 (e.g., the control loop of the system 2800) may still include a solid state temperature transducer (e.g., thermostat 1370), and a solid state humidistat (e.g., humidistat 1385) located at the system output. Additionally, as discussed in greater detail below, the system 2800 can include one or more Peltier modules (e.g., thermoelectric coolers) that help to adjust the temperature and the humidity of the incoming air.

The system 2800 may also include a microprocessor (e.g., microprocessor 360) that is programmed to maintain comfortable living conditions within the room/building, and control a number of the components of the system. For example, as also discussed above, the microprocessor 360 can control the speed of the chamber fan (e.g., fan 130). Additionally, the microprocessor 360 can control the duty cycle of the power to the heater/cooler 1380 (which may be a resistive heater) that helps temper the incoming air. Similarly, the microprocessor 360 can control the power to the Peltier module(s) located near the inlet of the system 2800. By controlling the power of the Peltier module(s), the microprocessor 360 can cool and/or dehumidify the incoming air.

In addition to controlling the power to various components of the system 2800, the microprocessor 360 can also serve a monitoring and alarm function. For example, the microprocessor 360 may monitor the power level of the active components (e.g., chamber fan 130, the heater 1380, the Peltier module(s), and the UV lamp(s) 210 (or a ballast for the UV lamps, if equipped)). The microprocessor 360 may then compare the power consumption of the active components with a stored value (or a stored power range) for the individual components and/or the system as a whole to validate that the system 2800 is functioning properly. If one or more of the components is not operating at the stored value (or within the stored power range), the microprocessor 360 can send a message that will be displayed on the control panel 330. Furthermore, by monitoring the power level of the fan 130, the microprocessor 360 will also be able to determine if the filters (e.g., filter 410, pre-filter 1027, pre-filter 1350, carbon filter 1390, etc.) are prematurely blocked due to high levels of collected airborne contamination.

The microprocessor 360 can also monitor and track the total system runtime. By tracking the total runtime, the microprocessor 360 is able to determine when it is time to change the cartridge 2860 (or a component of the cartridge 2860). For example, the microprocessor 360 can compare the total runtime with the expected life cycle of the components of the cartridge 2860. Once the microprocessor 360 determines that the runtime has reached a threshold value (e.g., a percentage of the expected life cycle), and that the cartridge 2860 needs to be changed, the microprocessor 360 sends a change cartridge signal that is displayed on the control panel 330. The user may then replace the cartridge 2860, as discussed above.

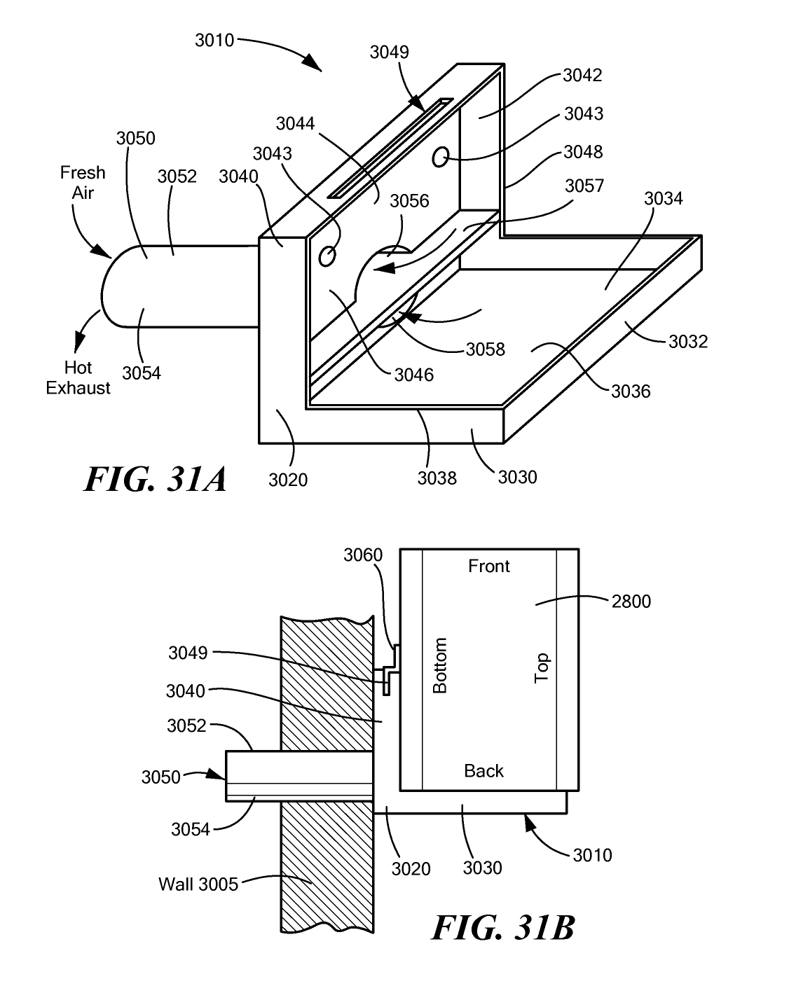

As mentioned above, some embodiments of the present invention may be installed into the window of a building. However, as shown in FIGS. 31A and 31B, to accommodate structures in which a window is not available, some embodiments may include a mounting kit 3010 that allows the system 2800 (or the other embodiments of the system) to be installed through the wall 3005 of the structure. The mounting kit 3010 may include an L-shaped body 3020 with a horizontal portion 3030 on which the system 2800 can sit, and a vertical portion 3040 that extends along the wall 3005. As best shown in FIG. 31A, both the horizontal portion 3030 and the vertical portion 3040 can have a wall 3032/3042 that extends from flat surface 3034/3044 to form a recess 3036/3046 in the horizontal and vertical portions 3030/3040. The recess 3036 within the horizontal portion 3030 can act as a condensate tray that collects condensate dripping from or otherwise forming on the system 2800. The wall 3032 on the horizontal portion 3030 and/or the wall 3042 on the vertical portion 3040 may have an edge gasket 3038/3048 that seals against the system 2800 to prevent leakage between the mounting kit 3010 and the system 2800.

Extending outward from the vertical portion 3040 (such that it may be installed through an opening in the wall 3005), the kit 3010 can include a divided tube 3050 that facilitates the flow of air between the atmosphere (e.g., the atmosphere outside of the building) and the system 2800. For example, the tube 3050 may be divided into an air inlet section/flowpath 3052 that allows air to be drawn into the system 2800 through a fresh air inlet 3056 within the vertical portion 3040 (e.g., extending through the flat surface 3044). Similarly, the tube 3050 may have an exhaust portion/flowpath 3054 that allows exhaust air (e.g., hot exhaust) to be vented/exhausted back to the outside atmosphere through an exhaust exit 3058 within the vertical portion 3040. Additionally, to prevent mixing of the incoming fresh air and the exhaust air, the kit 3010 includes a dividing plate 3057 between the inlet 3056 and the exhaust exit 3058. The plate 3057 may be insulated to prevent the hot exhaust from heating the incoming fresh air.

To secure the mounting kit 3010 to the wall 3005 (and subsequently secure the system 2800 to the mounting kit 3013), the user/installer may first prepare an appropriate sized hole through the wall 3005 (if one does not already exist) and pass the tube 3050 through the hole. The user/installer may then utilize mounting holes 3043 in the vertically extending portion 3040 to secure the mounting kit 3010 to the wall (e.g., using screws, bolts, or other attachment devices. Once the body 3020 is secured to the wall 3005 and the tube 3050 extends through the wall 3005, the user/installer may then secure the system 2800 to the mounting kit 3010. To that end, the system 2800 may include an attachment bracket 3060 (FIG. 31B) that extends along (and is secured to) a portion of the bottom of the system 2800. When securing the system 2800 to the mounting kit 3010, the user/installer may insert the attachment bracket 3060 into an attachment slot 3049 located on the vertical portion 3040, such that the back of the system 2800 rests on the horizontal portion 3030.

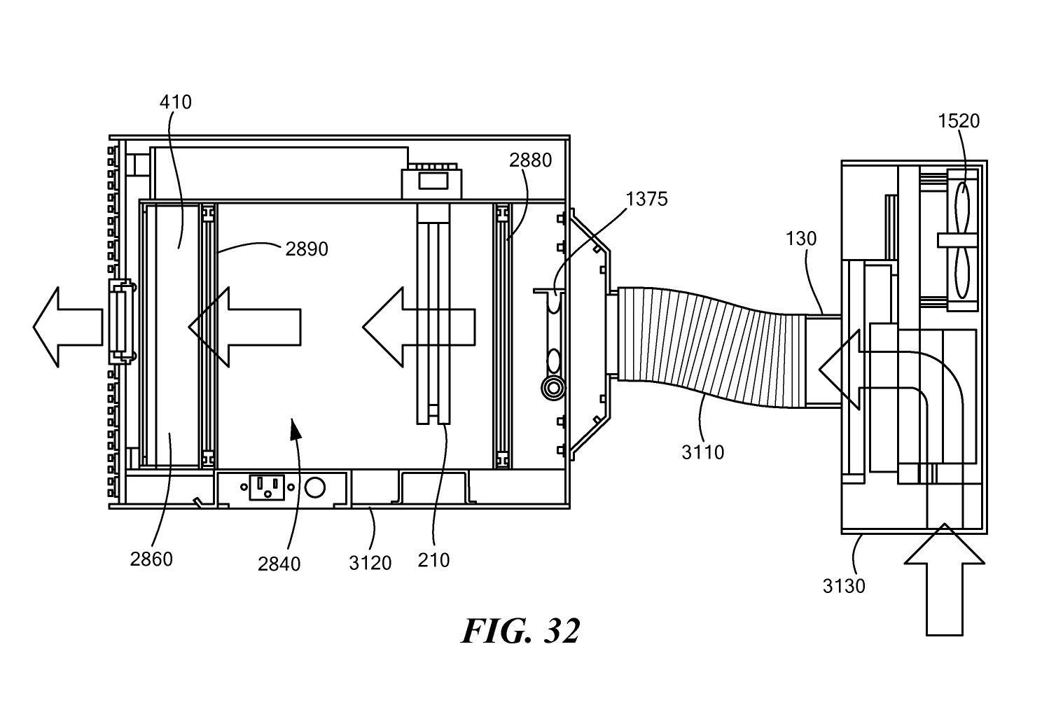

It is important to note that, although the above described embodiments have a single housing that houses all of the system components, in other embodiments, the system components can be separated. For example, as shown in FIG. 32, some of the air conditioning components (e.g., the Peltier modules, the resistive heater, etc.) can be separated from the air purification components (e.g., the germicidal radiation chamber 2840, the removable cartridge 2860, UV lamps 210, filters, electric heater 1375, etc.). In such embodiments the air conditioning components may be connected to the germicidal radiation chamber 2840 (and the remainder of the system) via a duct 3110 (e.g., a flexible duct) and an electrical cable. For example, the duct 3110 and cable allow the air conditioning components to be separated/removed from the air purification components and, perhaps, contained within a separate housing (e.g., housings 3120 and 3130). To that end, one section of the system (e.g., housing 3130) may be located in a window, and the other section (e.g., housing 3120) may be located within the interior of the house/building. To minimize components and the number of connections required, the duct 3110 may contain the cable (e.g., the cable may run through or along the duct).

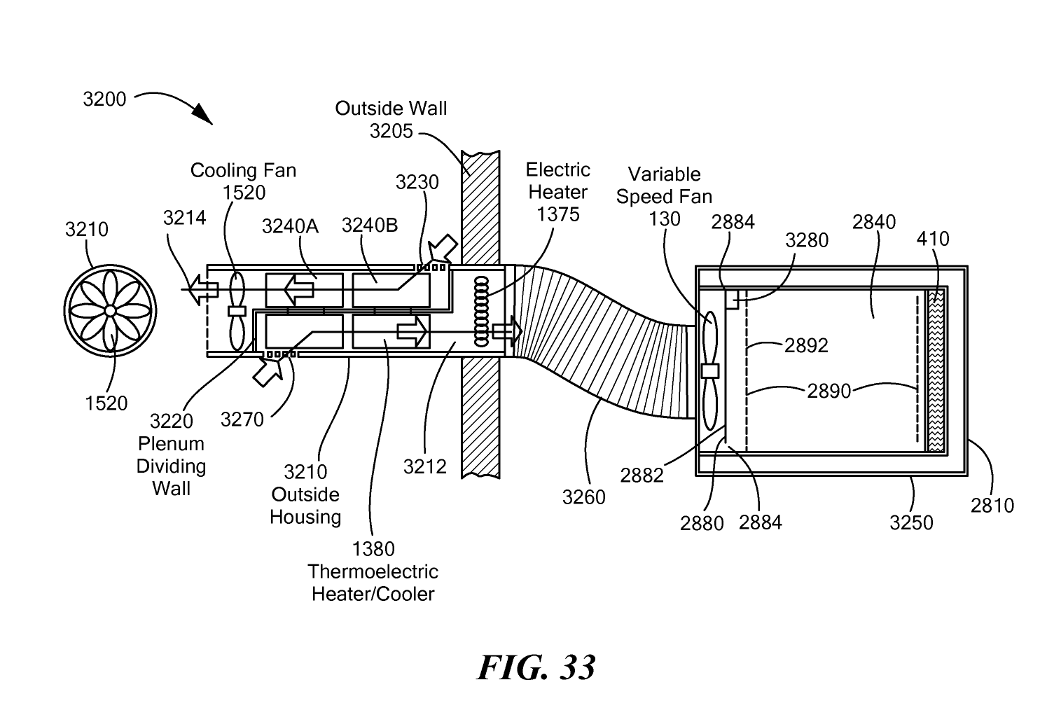

FIG. 33 shows an alternative embodiment of a two-part system that may be utilized in applications in which a window is not available for through-window installation. In such embodiments, the system 3200 may have an outside housing 3210 (e.g., with a round cross-sectional shape) that can extend through a hole in the wall 3205 of the structure (e.g., the home or building). The outside housing 3210 houses the cooling fan 1520, the Peltier modules 3240A/B, the thermoelectric heater/cooler 1380, and the electric heater 1375, and may also include a plenum dividing wall 3220 that divides the outside housing 3210 into two air flow paths. For example, the dividing wall 3220 may divide the outside housing 3210 into a primary air flow path 3212 (e.g., similar to the first air flow path 250 discussed above) and a secondary air for path 3214 (e.g., similar to the third air flow path 1510 discussed above). The cooling fan 1520 is located within the secondary air flow path, and when operational, draws outside air into the secondary air flow path through an opening 3230 near the wall 3205. This air flows over the hot side of the Peltier modules 3240A/B (e.g., to cool the Peltier modules 3240A/B), and is exhausted to the exterior of the structure through the end of the outside housing 3210.

Within the house/structure, the two part system 3200 may include an inside housing 3250 that houses the remaining components of the air-purification system. To connect the two housings, the system 3200 includes a flexible duct 3260 (with an electrical cable) that extends from the inside housing 3250 to the wall 3205 of the structure and the outside housing 3210. The inside housing 3250 houses the air purification equipment/component including, but not limited to, the germicidal radiation chamber 2840, the filter(s) 410 (and filters 1350/1390, if equipped), the variable speed fan 130, the baffles 2880/2890, the removable cartridge 2860, the UV lamps 210, etc. Additionally, like some of the systems described above, the inside housing 3250 may also have a front bezel 2810 (with a magnet) and a hall-effect transistor 2830 that shuts down the system 3200 if the bezel 2810 is removed.

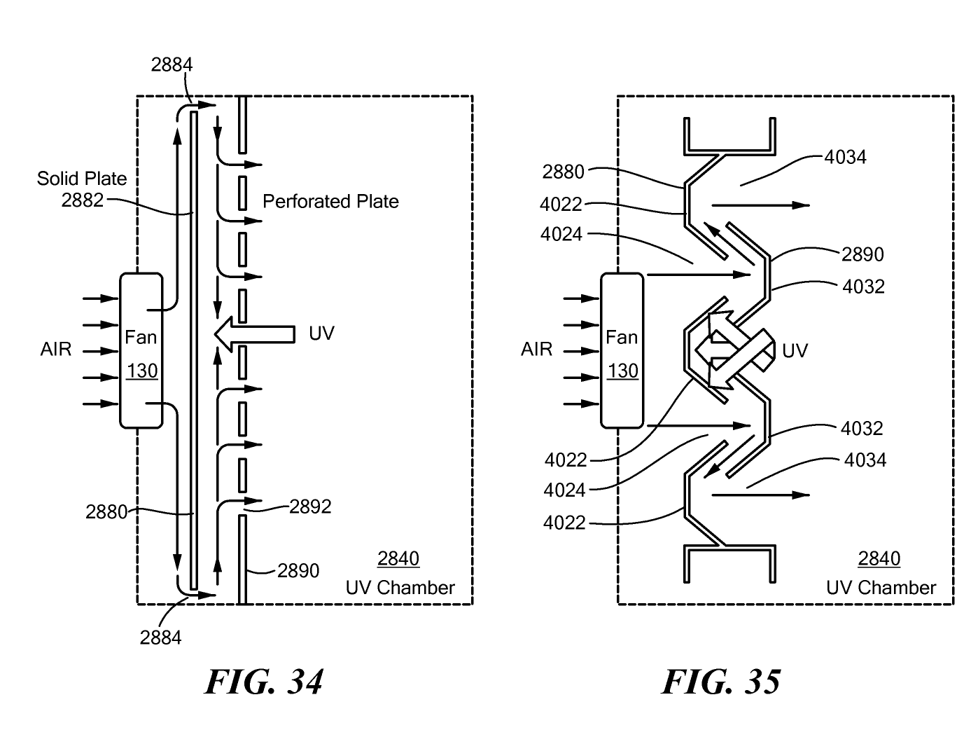

Although some embodiments can have a baffles/flow straightener configuration similar to that described above and shown in FIG. 30 (e.g., a perforated baffle 2880 and flow straightener 2890), other embodiments can utilize different baffle/flow straightener designs/configurations. For example, as shown in FIGS. 33 and 34, the baffle 2880 may be/include a flat plate 2882 and the flow straightener 2890 may be perforated plate 2892 located downstream of the flat plate 2882. To allow air to flow through the system 3200 (or system 2800 discussed above), the flat plate 2882 may not extend all the way to the edges/sides of the germicidal radiation chamber 2840, such that gaps 2884 are located/formed at one or more ends of the flat plate 2882. The air drawn into the system 3200 may flow through the gaps 2884, through the perforated flow straightener 2890, and into the main chamber 2852 of the germicidal radiation chamber 2840.

Alternatively, as shown in FIG. 35, the baffle/flow straightener configuration can have a more serpentine structure. For example, the baffle 2880 may include a number of angled sections 4022 (e.g., u-shaped sections) that are spaced from one another to form gaps 4024 between each of the angled sections 4022. Similarly, the flow straightener 2890 may also include a number of angled sections 4032 that are spaced from one another to form gaps 4034 between the angled sections 4032. To allow air to flow through the baffle 2880 and flow straightener 2890 and prevent UV light/radiation from exiting the radiation chamber 2840, the angled sections 4032 of the flow straightener 4030 may be aligned with the gaps 4024 between the angled sections 4022 of the baffle 4020 (e.g., angled sections 4022 may be offset from angled sections 4032).

During system operation, the variable speed fan 130 can draw air into the outside housing 3210 (e.g., into the primary air flow path 3212) through an air intake 3270 located upstream of the Peltier modules 3240A/B. As the air flows over the Peltier modules 3240A/B, thermoelectric heater/cooler 1380 and electric heater 1375, the system 3200 will condition the air as needed (e.g., as described above, it will dehumidify, heat, cool, etc. depending on what is needed to reach comfortable living conditions). The air will then flow through the flexible duct 3260 and into the inside housing 3250 where the air is purified as it passes through the filter(s) and germicidal radiation chamber 2840).

As discussed above, the microprocessor 360 can monitor the power level of the fan 130 to determine if the filters (e.g., filter 410, pre-filter 1027, pre-filter 1350, carbon filter 1390, etc.) are prematurely blocked due to high levels of collected airborne contamination. Additionally or alternatively, as shown in FIG. 33, some embodiments (e.g., the two-part system or the other embodiments described above) may have an air velocity sensor 3280 located within the germicidal radiation chamber 2840 (e.g., just downstream of the variable speed fan 130 and the baffle 2880). The air velocity sensor 3280 may be electrically connected to the microprocessor 360, and, as the air flows through the system 3200, the air velocity sensor 3280 may measure the velocity of the air flow (e.g., the velocity of the air passing over the baffle 2880). The microprocessor 360 can then monitor the measured velocity to determine if the filters (e.g., filter 410, pre-filter 1027, pre-filter 1350, carbon filter 1390, etc.) are blocked, for example, if the velocity drops below a threshold value. For example, if the velocity of the air flowing over the edges of the plate 3292 and through the gap(s) 3294 drops below a threshold (e.g., a stored value), then the microprocessor 360 will determine that one or more of the filters is blocked.

It is important to note that, in some atmospheric conditions, condensate may form within the system. For example, particularly during humid weather, condensate will form and drop of the cool side of the Peltier modules 3240A/B as the system 3200 conditions the incoming air. This condensate, if allowed to build up, may negatively impact the performance and longevity of the system. Therefore, in some embodiments, the outside housing 3210 may be tilted down slightly to allow for condensate within the outside housing 3210 to drain/drip out of the air intake 3270. Additionally, the electric heater 1375 may be placed near the wall 3205 (e.g., within the hole through the wall) to prevent cool air from cooling the walls of the flex tubing and forming condensate in the home/structure.

Additionally or alternatively, in some embodiments, the air conditioning components can run autonomously from the air purification components. In this manner, some embodiments can operate as an air conditioning unit, even if the air purification components are not operational, being replaced, or otherwise not being used.

Although various exemplary embodiments of the invention have been disclosed, it should be apparent to those skilled in the art that various changes and modifications can be made which will achieve some of the advantages of the invention without departing from the true scope of the invention. These and other obvious modifications are intended to be covered by the appended claims.

1. A system for reducing airborne contamination within a building comprising:
a housing defining the structure of the system;
a system inlet;
a system outlet;
a variable-speed fan configured to operate at a speed;
a microprocessor in communication with the fan and configured to control the speed of the fan;
an electrical chassis located within the housing and defining a chamber, the electrical chassis supporting at least some of the system's electrical components within the housing; and
a removable cartridge configured to be selectively coupled with the electrical chassis to form a germicidal radiation chamber within the housing and located within an airflow path through the system, the removable cartridge including at least one UV light source and at least one filter, the at least one UV light source and at least one filter configured to sterilize air as it passes through the system and the germicidal radiation chamber.

2. A system according to claim 1, wherein the removable cartridge includes a first electrical connector configured to connect with a second electrical connector located on the electrical chassis when the cartridge is coupled with the chassis, the first and second electrical connectors electrically connecting the microprocessor and the at least one UV lamp.

3. A system according to claim 2, wherein the microprocessor controls a power level of the at least one UV lamp.

4. A system according to claim 1, wherein the housing includes a removable bezel, the removable bezel removably connected to the housing via a magnet.

5. A system according to claim 4, further comprising a hall-effect transistor, a magnetic field created by the magnet energizing the hall-effect transistor when the bezel is connected to the housing.

6. A system according to claim 5, wherein removal of the removable bezel turns off the system.

7. A system according to claim 1, wherein the germicidal radiation chamber includes a perforated baffle at an input end of the germicidal radiation chamber.

8. A system according to claim 7, wherein the perforated baffle includes a titanium oxide coating.

9. A system according to claim 1, wherein the removable cartridge includes a perforated baffle.

10. A system according to claim 1, further comprising;
a temperature transducer located at the system outlet and configured to measure a temperature of air exiting the system; and
a humidistat located at the system outlet and configured to measure the humidity of air exiting the system.

11. A system according to claim 10, wherein the microprocessor is in electrical communication with the temperature transducer and the humidstat, the microprocessor configured to control system operation to maintain comfortable living conditions within the building, based at least in part on the measured temperature and the measured humidity.

12. A system according to claim 1, further comprising a resistive heater located at the system inlet, the resistive heater configured to temper incoming air.

13. A system according to claim 12, wherein the microprocessor is configured to control a duty cycle power to the resistive heater.

14. A system according to claim 1, further comprising a first Peltier module located at the system inlet, the first Peltier module configured to cool incoming air.

15. A system according to claim 14, further comprising a second Peltier module located at the system inlet, the second Peltier module configured to dehumidify incoming air.

16. A system according to claim 15, wherein the microprocessor is configured to control power to at least one of the first Peltier module and the second Peltier module.

17. A system according to claim 1, wherein the microprocessor is further configured to monitor the power consumption of the system and compare the monitored power consumption with a stored value to validate system functionality.

18. A system according to claim 17, further comprising a control panel having a display.

19. A system according to claim 18, wherein the microprocessor is further configured to send a message to the control panel indicating that that the system is operating out of specifications if the power consumption of the system is substantially not equal to the stored value.

20. A system according to claim 1, wherein the microprocessor is configured to monitor a total runtime of the system and send a change cartridge message to the control panel if the total runtime exceeds a threshold vale.

21. A system according to claim 1, wherein the microprocessor is further configured to monitor a power of level of the fan and determine, based at least in part on the power level of the fan, if the at least one filter is clogged.

22. A system according to claim 1, wherein the housing is configured to fit within a window of the building.

23. A system according to claim 1, further comprising:
a mounting kit configured to allow the housing to be secured to a wall of the building, the mounting kit having a body portion configured to support the housing and a divided tube configured to extend through an opening within the wall of the building.

24. A system according to claim 23, wherein the divided tube includes:
an air inlet pathway configured to allow the variable speed fan to draw air from an outside atmosphere through the air inlet pathway and into the system inlet; and
an exhaust pathway configured to allow the system to send exhaust air to the outside atmosphere.

25. A system according to claim 24, wherein the mounting kit further includes a dividing plate configured to separate the air inlet pathway from the exhaust pathway and prevent drawn air from mixing with exhaust air.

26. A system according to claim 25, wherein the dividing plate is insulated.

27. A system according to claim 23, wherein the mounting kit includes an attachment bracket configured to be attached to a surface of the housing and secure the housing to the body portion of the mounting kit.

28. A system according to claim 27, wherein the body portion of the mounting kit includes an attachment slot, at least a portion of the attachment bracket located within the attachment slot when the housing is secured to the body portion of the mounting kit.

29. A system according to claim 23, wherein the body portion of the mounting kit includes a vertically extending portion and a horizontally extending portion, the vertically extending portion configured to extend along a portion of the wall, the horizontally extending portion configured to support the housing.

30. A system according to claim 29, wherein the vertically extending portion includes mounting holes configured to allow the body portion of the mounting kit to be secured to the wall.

31. A system according to claim 29, wherein the horizontally extending portion forms a condensate tray.

32. A system according to claim 23, further comprising an edge gasket extending along at least a portion of the body portion, the edge gasket configured to seal against the housing.

33. A method for reducing airborne contamination within a building comprising:
installing an air purification system within an opening in the building, the air purification system including:
a housing defining the structure of the system;
a system inlet;
a system outlet;
a variable-speed fan configured to operate at a speed;
a microprocessor in communication with the fan and configured to control the speed of the fan;
an electrical chassis located within the housing and defining a chamber, the electrical chassis supporting at least some of the system's electrical components within the housing; and
a removable cartridge configured to be selectively coupled with the electrical chassis to form a germicidal radiation chamber within the housing and located within an airflow path in the system, the removable cartridge including at least one UV light source and at least one filter, the at least one UV light source and at least one filter configured to sterilize air as it passes through the system and the germicidal radiation chamber.
drawing air from outside the building through the system inlet and the airflow path, the germicidal radiation chamber sterilizing the air as it passes through the airflow path;
operating the air purification system at peak air flow; and
introducing the sterilized air into the building through the system outlet, the sterilized air displacing contaminated air within the building, the air pressure within the building increasing until the contaminated air displaced from the building equals a flow rate of the sterilized air entering the building.

34-65. (canceled)

66. A system for reducing airborne contamination within a building comprising:
an outside housing configured to extend through a wall of the building;
an inside housing configured to be located within the interior of the building;
a flexible duct extending between the outside housing and inside housing and configured to allow air to flow from the outside housing to the inside housing
a system inlet located within the outside housing;
a system outlet located within the inside housing;
a variable-speed fan located within the inside housing and configured to operate at a speed to draw air into the system inlet;
a microprocessor in communication with the fan and configured to control the speed of the fan;
an electrical chassis located within the inside housing and defining a chamber, the electrical chassis supporting at least some of the system's electrical components within the inside housing; and
a removable cartridge configured to be selectively coupled with the electrical chassis to form a germicidal radiation chamber within the inside housing and located within a main airflow path through the system, the removable cartridge including at least one UV light source and at least one filter, the at least one UV light source and at least one filter configured to sterilize air as it passes through the system and the germicidal radiation chamber.

67-97. (canceled)
